(19) United States
(12) Patent Application Publication
(10) Pub. No.: US 2007/0116762 A1
(43) Pub. Date: May 24, 2007

(54) Compositions of stabilized ramipril in combination with another active agent

(76) Inventors: Edward S. Wilson, Cary, NC (US); Kevin H. Sills, Apex, NC (US); Martin W. Beasley, Cary, NC (US); David P. Hause, Carrboro, NC (US)

Correspondence Address: JONES DAY, 222 EAST 41ST ST, NEW YORK, NY, 10017, US

(21) Appl. No.: 11/594,535

(22) Filed: Nov. 7, 2006

Related U.S. Application Data
(60) Provisional application No. 60/736,947, filed on Nov. 7, 2005.

(51) Int. Cl. A61K 31/549; A61K 31/403; A61K 9/20

(52) U.S. Cl. 424/464; 514/223.5; 514/412

(57) ABSTRACT

A pharmaceutical composition comprising ramipril, another active agent, and a blending agent, wherein in the ramipril is coated by the blending agent, and wherein the blending agent is glyceryl behenate, glyceryl stearate, stearyl alcohol, macrogol stearate ether, palmitosearate, ethylene glycol, polyethylene glycol, stearic acid, cetyl alcohol, lauryl alcohol, amylopectin, poloxymer or combinations thereof.

This application claims the benefit of U.S. Provisional Application No. 60/736,947, filed Nov. 7, 2005, the contents of which are incorporated herein in its entirety.

### FIELD OF THE INVENTION

The present invention relates to novel pharmaceutical compositions comprising ramipril in combination with other active agents. More particularly, the compositions of the present invention have improved stability of ramipril which is less susceptible to degradation relative to other compositions comprising ramipril alone or in combination with another active agent. The present invention also relates to methods of making and methods of manufacturing such compositions.

### BACKGROUND

Cardiovascular disease treatment has evolved rapidly over the last few decades to include agents that range in diversity from diuretics and natural products such as rauwolfia serpentina to agents such as angiotensin converting enzyme (ACE) inhibitors and calcium channel blockers (CCB). In efforts to achieve improved therapy (primarily for the treatment of hypertension, its sequelae, reversible conditions secondary to hypertension, and hypertension secondary to other conditions), a number of agents have been tested both alone as well as in combination with other agents. Some of the conditions for which at least one of these agents has been used or is believed useful include, without limitation, hypertension, angina, myocardial infarction, atherosclerosis, diabetic nephropathy, diabetic cardiac myopathy, renal insufficiency, peripheral vascular disease, left ventricular hypertrophy, cognitive dysfunction, stroke, and headache. Indeed, many agents useful for the treatment of cardiovascular disease and related conditions are apparent to those of ordinary skill in the art based on a knowledge of the underlying mechanisms involved in certain conditions as well as on general clinical and pre-clinical experience (U.S. Pat. No. 6,162,802, issued Dec. 19, 2000 to Papa, et al.).

Because the treatment of cardiovascular disease has become complex and can often entail the administration of multiple agents, there is a need for combination products. The incorporation of two or more agents useful in the treatment of cardiovascular disease into one single unit dosage form can eliminate the need to take multiple single agent dosage forms. Combination products can simplify the dosing schedule and thereby improve patent compliance and diminish the need for multiple administrations or multiple single agent dosage forms.

Although it is generally recognized that combination therapy can be more efficiently affected using combination products that incorporate two or more active agents, there are practical hurdles to formulating active agents into a single combination product.

For example, although the usefulness of having a combination product containing an inhibitor of the renin-angiotensin system or a pharmaceutically acceptable derivative thereof and an other antihypertensive, a cholesterol lowering agent, a diuretic or aspirin for the use in the prevention of cardiovascular events is suggested in U.S. patent application Publication No. 2005/0101658, published May 12, 2005 (Scholkens et al.), the publication does not teach or suggest any tablets containing ramipril in combination with another active agent.

Likewise, although the combination of an angiotensin II antagonist with drugs selected from among the remedies for hypertension, hypoglycemics, hyperlipemia, antithromboties, menopause and anticancer drugs is suggested in U.S. patent application Publication No. 2004/0219208, published Nov. 4, 2004 (Kawamura, et al.) the publication does not teach or suggest any tablets containing ramipril in combination with another active agent.

In general, drug stability is an important consideration during the design, manufacture and storage of pharmaceutical compositions. Drugs that lack stability can degrade into degradant products which can cause side effects or, in some instances, can cause a decrease in the efficacy and bioavailability of the drug itself, making it difficult for doctors to prescribe consistent and effective treatments. This is applicable to the formulation of combination products and can be even more complex because of the inherent difficulties that accompany formulating combination products.

Oftentimes, the incompatibility of active agents can make the process of formulating the agents into a single unit dosage form very difficult. In certain instances, the potency, stability, and/or bioavailability of one or more of the agents is adversely affected in comparison to the single agent counterparts. Indeed, it is known that ACE inhibitors, a class of drugs that is extremely useful in the treatment of cardiovascular disease, are susceptible to degradation, particularly when subjected to the stresses inherent to formulation processes.

Ramipril is an ACE inhibitor used in the treatment of cardiovascular disease, especially hypertension, and it is one of the most frequently prescribed drugs for congestive heart failure. In hypertensive patients, ramipril is known to reduce peripheral arterial resistance causing a reduction in blood pressure without a compensatory rise in heart rate. Ramipril has also been shown to reduce mortality in patients with clinical signs of congestive heart failure after surviving an acute myocardial infarction. Ramipril may have an added advantage over many other ACE inhibitors due to its pronounced inhibition of the ACE enzymes in tissues resulting in organ protective effects in such organs as the heart, kidney, and blood vessels.

Even though ramipril is without question one of the most important ACE inhibitors available today, like other ACE inhibitors, ramipril is susceptible to degradation. Indeed, current ramipril formulations show a considerable degree of instability. To date, the leading formulation of ramipril is a capsule. The degradation of ramipril is believed to occur mainly via two pathways: (a) hydrolysis to ramipril-diacid; and (b) cyclization or condensation to ramipril-diketopiperazine, also referred herein as ranipril-DKP. These ramipril-diacid and ramipril-DKP compounds form, as indicated above, as a result of cyclization, condensation and/or breakdown arising from exposure to heat, air, moisture, stress, compaction or other interactions or events.

Indeed, ramipril needs special care when formulating into pharmaceutical preparations due the physical stress associated with the formulation process. Factors that influence the stability of ramipril formulations are mechanical stress, compression, manufacturing processes, excipients, storage conditions, heat and moisture. In particular, the physical stress associated with formulating tablets can increase the decomposition of ramipril into degradant products. Moreover, addition of other active agents to a ramipril tablet formulation could further increase the rate of ramipril decomposition and affect the potency or bioavailability of ramipril. In certain instances, the potency or bioavailability of the other active agent can be adversely affected by the presence of ramipril in the formulation or during the formulation process.

Attempts to overcome ramipril stability have been reported in PCT/EP2004/00456 and PCT/CA2002/01379.

PCT/EP2004/00456 describes solid ramipril compositions having suitably low water content and a process that utilizes excipients with low water content, in combination with processing parameters and packaging material that prohibit water or moisture uptake to formulate ramipril compositions and even though some formulations use glyceryl dibehenate, the rate of ramipril-DKP formulation is much higher than that in present invention. After one month the percent weight of ramipril-DKP is 2.14% at 40° C. and at 75% humidity.

PCT/CA2002/01379 describes solid ramipril compositions that comprise a mixture of ramipril and lactose monohydrate as the diluent. According to PCT/EP2004/000456, the process includes lactose monohydrate as the major excipient to formulate ramipril compositions in its attempt to improve ramipril stability. However, with the lactose monohydrate, the lowest rate of ramipril-DKP formation shows the present of ramipril-DKP at 1.10%, immediately after formation of the capsule.

U.S. patent application Publication No. 2005/0069586, published Mar. 31, 2005 (Hrakovsky, et al.) describes ramipril tablets that have an admixture of ramipril and sodium stearyl fumerate with reduced ramipril-DKP formation, but does not teach pre-blending or co-milling the ramipril with glyceryl behenate or substantially coating the ramipril with any blending agent.

As such, there remains a need for formulations, in particular oral dosage forms such as tablets that contain ramipril in combination with at least one other active agent, wherein the ramipril does not degrade and maintains its potency under formulation and storage conditions. There also remains a need for formulations that contain ramipril in combination with one or more active agents wherein the bioavailability of ramipril and the other agent is the same or improved in comparison to the single agent forms.

Citations of any reference in the Background section of this application is not an admission that the reference is prior art to the application.

### SUMMARY OF THE INVENTION

The present invention is based in part on the discovery that stable oral dosage forms comprising ramipril and at least one other active agent can be achieved by first pre-blending or co-milling glyceryl behenate with ramipril during manufacture of oral dosage forms that contain ramipril and another active agent. In particular, the inventors have made the surprising discovery that by combining ramipril with glyceryl behenate, prior to formulation of ramipril and a diuretic into a tablet dosage form, the rate of ramipril degradant production is extremely low. In fact, it is particularly surprising that even when the formulation contains another active agent, the potency and stability of ramipril in the compositions of the subject invention is improved compared to current ramipril formulations. The inventors have also discovered that the bioavailability of the ramipril and the other agent of the combination tablet remain effectively the same as compared to the bioavailability of single agent tablets. Without being limited to one particular theory, the inventors of the present invention believe that the glyceryl behenate coats the ramipril and is able to protect the ramipril from physical and environmental stress that, under normal conditions, cause the ramipril to degrade into degradant products such as ramipril-DKP and ramipril-diacid.

In particular, the inventors have demonstrated that by utilizing glyceryl behenate as a blending agent, ramipril decomposition into degradant products, such as ramipril-DKP and ramipril diacid, can be significantly reduced. Indeed, the inventors have demonstrated that the rate of decomposition of ramipril in compositions of the invention is less than 0.05% of the total weight of ramipril on average per month for at least 36 months from the date that the ramipril compositions are first formulated. Moreover, the inventors have demonstrated that the ramipril in the tablets of the invention containing ramipril and chlorthalidone is as bioavailable as ramipril formulated alone

As such, the pharmaceutical compositions contemplated by the present invention comprise ramipril in combination with at least one other active ingredient, wherein the ramipril has a low rate of degradation and is substantially free of ramipril-DKP and ramipril-diacid. Moreover, the pharmaceutical compositions of the present invention have increased stability, bioavailability and shelf-life compared to current formulations comprising ramipril alone. Additionally, the pharmaceutical compositions of the present invention allow ramipril to maintain potency, assuring health care providers and patients that they are giving and receiving consistent and exact treatment. The invention also contemplates reducing the rate of ramipril-DKP formation, especially under formulation and extended storage conditions.

The present invention also relates to methods of making the pharmaceutical compositions, of the present invention. Such methods comprise first pre-blending and/or co-milling ramipril with a blending agent before combining with other active agents and excipients. The methods of the present invention also comprise first coating ramipril with a blending agent prior to formulation of ramipril into a dosage form that includes one or more additional active agents.

### BRIEF DESCRIPTION OF THE FIGURES

FIG. 1—Method of making tablets

FIG. 2—Mean Whole Blood Chlorthalidone Concentrations Versus Time (Linear Scale), Group I of Example 2

FIG. 3—Mean Whole Blood Chlorthalidone Concentrations Versus Time (Linear Scale), Group II of Example 2

FIG. 4—Mean Whole Blood Chlorthalidone Concentrations Versus Time (Linear Scale), Group III of Example 2

FIG. 5—Mean (SD) Plasma Ramipril Concentrations Versus Time for Group I of Example 3 (Linear Scale)

FIG. 6—Mean (SD) Plasma Ramipril Concentrations Versus Time for Group II of Example 3 (Linear Scale)

FIG. 7—Mean (SD) Plasma Ramipril Concentrations Versus Time for Group III of Example 3 (Linear Scale)

FIG. 8—Mean (SD) Plasma Ramipril Concentrations Versus Time for Group IV of Example 3 (Linear Scale)

FIG. 9—Mean (SD) Plasma Ramipril Concentrations Versus Time for Groups I-IV of Example 3 (Linear Scale)

### DETAILED DESCRIPTION

The terms “stabilized”, “stability”, “improved stability” or “stable” as applied to ramipril, can encompass products that are substantially free of breakdown products or degradants. Such products or degradants include, but are not limited to, ramipril-diacid and ramipril-DKP.

The term “substantially free” refers to the ramipril formulations described herein that have significantly reduced levels of detectable breakdown products; e.g., ramipril-diacid and/or ramipril-DKP.

The term “cardiovascular disorder(s)” as used herein broadly and encompasses any disease, illness, sickness, disorder, condition, symptom or issue involving or concerning any part or portion of the heart or blood vessels of an animal, including a human. The term “blood vessel”, as used herein, is defined to include any vessel in which blood circulates. Such cardiovascular disorders include, for example, arterial enlargements, arterial narrowings, peripheral artery disease, atherosclerotic cardiovascular disease, high blood pressure, angina, irregular heart rates, inappropriate rapid heart rate, inappropriate slow heart rate, angina pectoris, heart attack, myocardial infarction, transient ischemic attacks, heart enlargement, heart failure, congested heart failure, heart muscle weakness, inflammation of the heart muscle, overall heart pumping weakness, heart valve leaks, heart valve stenosis (failure-to-open fully), infection of the heart valve leaflets, heart stoppage, asymptomatic left ventricular dysfunction, cerebrovascular incidents, strokes, chronic renal insufficiency, and diabetic or hypertensive nephropathy. These above-listed conditions commonly arise in healthy, pre-disposed or critically ill patients, and may or may not be accompanied by hypertension, angina, light-headedness, dizziness, fatigue or other symptoms.

The terms “treat(s)”, “treated”, “treating” or “treatment” are used herein interchangeably and refer to any treatment of a disorder in an animal diagnosed or inflicted with such disorder and includes, but is not limited to: (a) caring for an animal diagnosed or inflicted with a disorder; (b) curing or healing an animal diagnosed or inflicted with a disorder; (c) causing regression of a disorder in an animal; (d) arresting further development or progression of a disorder in an animal; (e) slowing the course of a disorder in an animal; (f) relieving, improving, decreasing or stopping the conditions of a disorder in a animal; (g) relieving, decreasing or stopping the symptoms caused by or associated with a disorder in an animal; or (h) reducing the frequency, number or severity of episodes caused by or associated with a disorder in an animal.

The terms “prevent(s)”, “prevented”, “preventing” or “prevention” are used herein interchangeably and refer to any prevention or any contribution to the prevention of a disorder in an animal or the development of a disorder if none has occurred in an animal which may be predisposed to such disorder but has not yet been inflicted with or diagnosed as having such disorder.

The phrase “safe and effective amount(s)”, as used herein, means any amount of a drug which, when administered to a subject to be treated, will achieve a beneficial pharmacological effect or therapeutic improvement consistent with the objectives of the present invention without causing serious, adverse or otherwise treatment-limiting side effects (at a reasonable benefit/risk ratio), within the scope of sound medical judgment.

The term “about” as used herein means approximately or near or around. For example, when the term “about” is used in relation to a specified dosage amount or range, the term “about” indicates that the dosage amount or range specified is an approximate dosage amount or range and that it includes not only the amount or range actually specified, but those amounts or ranges that may also be safe and effective amounts that are somewhat outside the cited amount or range.

As used herein, the terms “comprising,” “comprises”, “comprised of,” “including,” “includes,” “included,” “involving,” “involves,” “involved,” and “such as” are used in their open, non-limiting sense.

It should be understood that the phrase “pharmaceutically acceptable” is used adjectivally herein to mean that the modified noun is appropriate for use in a pharmaceutical product.

The term “pharmaceutically acceptable salt” refers to a salt that retains the biological effectiveness of the free acid and/or base of the specified compound. Examples of pharmaceutically acceptable salts include sulfates, pyrosulfates, bisulfates, sulfites, bisulfites, phosphates, monohydrogenphosphates, dihydrogenphosphates, metaphosphates, pyrophosphates, chlorides, bromides, iodides, acetates, propionates, decanoates, caprylates, acrylates, formates, isobutyrates, caproates, heptanoates, propiolates, oxalates, malonates, succinates, suberates, sebacates, fumarates, maleates, butyne-1,4-dioates, hexyne-1,6-dioates, benzoates, chlorobenzoates, methylbenzoates, dinitrobenzoates, hydroxybenzoates, methoxybenzoates, phthalates, sulfonates, xylenesulfonates, phylacetates, phenylpropionates, phenylbutyrates, citrates, lactates, gamma-hydroxybutyrates, glycollates, tartarates, methane-sulfonates, propanesulfonates, naphthalene-1-sulfonates, naphthalene-2-sulfonates, and mandelates. Several of the officially approved salts are listed in Remington: The Science and Practice of Pharmacy, Mack Publ. Co., Easton. Additional examples of pharmaceutically acceptable salts are besylate, edisylate, and mesylate salts (Gould, Philip L, 1986, “Salt selection for basic drugs”, Int. J. Pharmaceutics, 33, pp 201-217).

The term “derivative” as used herein means a chemically modified compound wherein the chemical modification takes place at one or more functional groups of the compound and/or on an aromatic ring, when present. The derivative may or may not retain the pharmacological activity of the compound from which it is derived.

The term “pharmaceutical grade” as used herein, means that a substance meets pharmaceutical standards, and that its purity is superior as compared to the purity of the same such substance when classified as food grade, which is less pure.

In general, the pharmaceutical compositions of the present invention relate to compositions comprising a combination of two or more drugs wherein at least one of the drugs is susceptible to degradation when exposed to the environment or exposed to physical stresses during the manufacturing process and wherein the rate of degradation of the compound is extremely low.

The present invention encompasses pharmaceutical compositions that comprise a combination of two or more drugs wherein at least one of the drugs is susceptible to degradation when exposed to the environment or exposed to physical stresses during the manufacturing process; and a blending agent.

In certain preferred embodiments, the drug susceptible to degradation is an ACE inhibitor. Suitable ACE inhibitors include, but are not limited to, captopril, benazepril, enalapril, lisinopril, fosinopril, ramipril, perindopril, quinapril, moexipril, and trandolapril

Of the ACE inhibitors, ramipril, its derivatives and salts are of special interest. Suitable ramipril derivatives and salts include, but are not limited to, the esters and those common salts known to be substantially equivalent to ramipril. Suitable ramipril esters include, but are not limited to, hexahydroramipril, ramipril benzyl ester, isopropyl ester, ethyl ester or methyl ester. Pharmaceutically acceptable salts of ramipril include, but are not limited to, salts with pharmaceutically acceptable amines or inorganic or organic acids such as, HCl, HBr, H₂SO₄, maleic acid, fumaric acid, tartaric acid and citric acid. Additional examples of pharmaceutically acceptable salts are besylate, edisylate, and mesylate salts.

Ramipril is a 2-aza-bicyclo [3.3.0]-octane-3-carboxylic acid derivative with five chiral centers, and 32 different enantiomeric forms. The chemical name of ramipril is (2S,3aS,6aS)-1[(S)—N—[(S)-1-carboxy-3-phenylpropyl]alanyl]octahydrocyclo-penta[b]pyrrole-2-carboxylic acid, 1-ethyl ester is most preferred and has the following chemical structure:

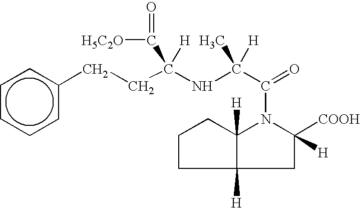

Ramipril is converted to ramiprilat in the body by hepatic cleavage of the ester group. Ramiprilat, the diacid or free acid metabolite of ramipril, is obtained in vivo upon administration of ramipril but ramiprilat is not absorbed appreciably in-vivo from the GI tract.

In preferred embodiments of the present invention the percent of ramiprilat does not exceed 20% after 8 weeks at 40° C. and 75% relative humidity. Preferably, the percent of ramiprilat does not exceed 1.0% during the life of the composition. Most preferably, the percent of ramiprilat does not exceed 0.5% during the life of the composition.

Ramipril is marketed in the United States under the brand name Altace® and abroad under the brand name Delix®. Altace® (ramipril) is supplied as hard shell capsules for oral administration containing 1.25 mg, 2.5 mg, 5 mg or 10 mg of ramipril.

Ramipril compositions of the present invention can be formulated with any form of ramipril known in the art. Ramipril suitable for the present invention can be uncoated or be coated with a coat forming material. Ramipril and processes for making and using ramipril are described and claimed in U.S. Pat. Nos. 4,587,258, 5,061,722 and 5,403,856, all of which are incorporated herein by reference in their entirety. The preparation of ramipril has also been described in EP 0 079 022 A2, EP 0 317 878 A1 and DE 44 20 102 A, which are incorporated herein by reference in their entirety.

Uncoated ramipril suitable for the present invention includes ramipril, as obtained from sanofi-aventis (Paris, France). Coated ramipril suitable for the present invention can be any coated ramipril known in the art. For example, coated ramipril suitable for the present invention can include ramipril particles that are coated with a suitable coat forming material. Coated ramipril suitable for the present invention can be partially, substantially or completely covered with a coat forming material. Ramipril particles can include but are not limited to, coated ramipril micro- or nanoparticles, coated ramipril crystalline particles, coated individual ramipril crystals and coated ramipril agglomerates, granules or beads. One preferred type of ramipril agglomerates is the GEcoated ramipril agglomerates, manufactured by Aventis Pharma Deutschland GmbH (Frankfurt on Main, Germany). Such GEcoated ramipril agglomerates are ramipril agglomerates coated with a hydroxypropyl methylcellulose polymer coating (1.192 mg GEcoated granules=1.0 mg ramipril). Coated ramipril particles, suitable for the present invention, can also be made according to the methods disclosed in U.S. Pat. Nos. 5,061,722; 5,151,433; 5,403,856; and 5,442,008, U.S. Provisional Application No. 60/625,270 and co-pending U.S. applications U.S. Ser. No. 11/269,387, filed Nov. 7, 2005 (published as U.S. patent application Publication No. 2006-0159742) and U.S. Ser. No. 11/269,388, filed Nov. 7, 2005 (published as U.S. patent application Publication No. 2006-0134213), herein incorporated by reference. The compositions of the present invention can also contain anhydrous, pharmaceutical grade ramipril powder comprising coated ramipril particles.

Accordingly, in preferred embodiments, the pharmaceutical compositions of the present invention comprise ramipril in combination with at least one other active agent, wherein the ramipril is substantially stable against decomposition into degradant products, such as ramipril-diacid and ramipril-DKP. Additionally, the ramipril compositions of the present invention have improved stability and shelf-life. This improved stability allows the ramipril compositions to maintain potency and improve effectiveness and bioavailability of ramipril compared to other ramipril formulations.

In the various embodiments contemplated, non-limiting examples of the active agents that can be formulated in combination with ramipril include: diuretics such as but not limited to chlorthalidone, furosemide, bumetanide, torsemide, hydrochlorothiazide, metolazone, and spironolactone; angiotensin receptor blockers such as but not limited to candesartan, eprosartan, irbesartan, telmisartan, valsartan and losartan; other ACE inhibitors such as but not limited to captopril, benazepril, enalapril, lisinopril, fosinopril, perindopril, quinapril, moexipril and trandolapril; cholesterol lowering drugs such as but not limited to atorvastatin, fluvastatin, lovastatin, pravastatin, rosuvastatin and simvastatin; calcium channel blockers such as but not limited to amlodipine, felodipine, dilitiazem, verapamil, nifedipine, nicardipine, nisoldpine and bepridil; beta blockers; glucose lowering agents such as but not limited to insulin, and oral hypoglycemics such as but not limited to the sulfonylurea class (i.e., metformin).

In preferred embodiments, the diuretics contemplated in the present invention are chlorthalidone and hydrochlorthiazide.

Chlorthalidone is a monosulfamyl diuretic that differs chemically from thiazide diuretics in that a double ring system is incorporated in its structure. It is 2-chloro-5-(1-hydroxy-3-oxo-1-isoindolinyl) benzenesulfonamide with an molecular formula of C₁₄H₁₁ClN₂O₄S, a molecular weight of 338.76 and the following structural formula:

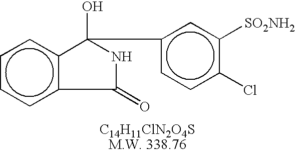

Hydrochlorothiazide is a diuretic and antihypertensive. It is the 3,4-dihydro derivative of chlorothiazide. Its chemical name is 6-chloro-3,4-dihydro-2H-1,2,4-benzothiadiazine-7-sulfonamide 1,1-dioxide. Its empirical formula is C₇H₈ClN₃O₄S₂ and its structural formula is:

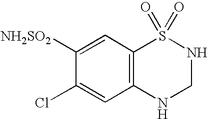

In various embodiments of the invention, the compositions comprise ramipril and a diuretic, wherein the bioavailability of the ramipril is the same as the bioavailability of ramipril when formulated alone. In certain preferred embodiments, the potency and stability of ramipril is improved when compared to current ramipril formulations. Moreover, in certain embodiments, the bioavailability or potency of the diuretic is improved or the same when compared to current single agent diuretic formulations.

The rate of decomposition of ramipril to ramipril-DKP, in the compositions of the present invention is between 0.00-0.11% of the total weight of ramipril per month. Preferably the rate of decomposition of ramipril in the compositions of the present invention is 0.04-0.095% of the total weight of ramipril per month.

For example, the ramipril compositions of the present invention result in ramipril-DKP formation of between about 0.0-0.6% or 0.0-0.6% of the total weight of ramipril during about the first three months or the first three months after the compositions are formed and between about 0-4% or 0-4% of the total weight of ramipril during a period of at least about 36 months or 36 months after the composition are formed.

In one embodiment, the pharmaceutical compositions of the present invention have a rate of decomposition of ramipril of less than about 0.04% to about 0.095% of the total weight of ramipril at room temperature or less than 0.04% to 0.095% of the total weight of ramipril at room temperature, on average per month for at least about 36 months or at least 36 months from the date that the ramipril compositions are first formulated. Preferred pharmaceutical compositions have ramipril-DKP formation of less than about 0.04% to about 0.085% or less than 0.04% to 0.085% of the total weight of ramipril at room temperature, on average per month for an extended period, more preferred the pharmaceutical compositions have ramipril-DKP formation of less than about 0.04% to about 0.055% or less than 0.04% to 0.055% of the total weight of ramipril at room temperature, per month on average for such an extended period, and even more preferred the pharmaceutical compositions have ramipril-DKP formation of less than about 0.04% to about 0.042% or less than 0.04% to 0.042% of the total weight of ramipril at room temperature, per month on average for an extended period of time.

Preferably the compositions of the present invention result in ramipril-DKP formation of less than about 0.3% or less than 0.3% during about the first three months of the total weight of ramipril and less than about 2.0% or less than 2.0% of the total weight of ramipril during a period of at least about 36 months after the first three month period. Preferred compositions result in ramipril-DKP formation of less than about 0.3% or less than 0.3% of the total weight of ramipril during about the first three months and less than about 1.5% or less than 1.5% of the total weight of ramipril during a period of at least about 36 months after the first three month period.

In one preferred embodiment, the compositions of the present invention comprise ramipril in combination with at least one other active agent, wherein the rate ramipril decomposition to ramipril-DKP, is less than about 0.3% or less than 0.3% of the total weight of the ramipril during about the first three months after the compositions are formed.

In another preferred embodiment, the compositions of the present invention comprise ramipril in combination with at least one other active agent, wherein the rate of ramipril decomposition to ramipril-DKP, is less than about 0.75% or less than 0.75% of the total weight of the ramipril during about the first 6 months after the compositions are formed.

In yet another preferred embodiment, the compositions of the present invention comprise ramipril in combination with at least one other active agent, wherein the rate of ramipril decomposition to ramipril-DKP, is less than about 3.0% or less than 3.0% of the total weight of the ramipril during about the first 36 months after the compositions are formed.

In all of the above embodiments, the active agent formulated in combination with ramipril is preferably a diuretic. In such embodiments, the bioavailability and/or potency and stability of the diuretic is the same or improved when compared to single agent formulations.

The blending agent in each of the compositions of the invention can be any substance suitable for pre-blending and co-milling, which stabilizes the drug and significantly reduces the degradation of the drug. The phrase “blending agent” is interchangeable with “blending compound”. Preferably, the blending agent can coat the ramipril and reduce the degradation rate.

Blending agents contemplated by the present invention include polymers, starches, stearates, silicas, waxes (atomized glyceryl palmitostearate, dioctyl sodium sulphosuccinate), surfactants, and fatty acids (preferably having a chain length of eight carbons or greater which may contain one or more double bonds). For example, blending agents suitable for the present invention include, but are not limited to, include long chain fatty acid-containing glycerol esters. Blending agents include, but are not limited to, glyceryl behenate, glyceryl stearate, stearyl alcohol, magnesium stearate, macrogol stearate ether, palmitosearate, ethylene glycol, polyethylene glycol, ethylene oxide polymers, sodium lauryl sulfate, magnesium lauryl sulfate, sodium oleate, sodium stearyl fumerate, leucine, stearic acid, cetyl alcohol, lauryl alcohol, amylopectin, poloxymer or combinations thereof. Most preferably, the blending agent is glyceryl behenate.

The blending agent can be present from at least about 0.1 wt % or from at least about 0.1 wt % and above by weight of the total composition. In a specific embodiment, the blending agent is present at about 0.5 wt. % and above or 0.5 wt. % and above. In another specific embodiment, the blending agent is present at about 1.0 wt. % and above or 1.0 wt. % and above. In another specific embodiment, the blending agent is present at about 2.0 wt. % and above or 2.0 wt. % and above. In a specific and preferred embodiment, the blending agent is present at about 3.0 wt. % and above or 3.0 wt. % and above. In another specific embodiment, the blending agent is present at about 4 wt. % and above (e.g., 5 and 10 wt. %).

Additionally, the blending agent can be present in a ratio of about 1:10 to about 10:1 or in a ratio of 1:0 to 10:1 of the drug. The blending agent can be present in a ratio of about 1:5 to about 5:1 or in a ratio of 1:5 to 5:1 or about 1:2 or 2:1 or in a ratio of 1:2 to 2:1 of the drug.

In yet another embodiment, the pharmaceutical compositions of the present invention comprise ramipril and a blending agent in combination with at least one other active agent, wherein ramipril is coated by the blending agent. In certain embodiments ramipril can be substantially coated by the blending agent. The ramipril is substantially coated when the blending agent coats ramipril wherein ramipril has a low or no rate of degradation. For example, the ramipril can be between about 50% to 100% or between 50% to 100% coated by the blending agent. Preferably, the ramipril is between about 75% to 100% or between 75% to 100% coated by the blending agent or more preferably between about 85% to 100% or between 85% to 100% coated by the blending agent. Most preferably, the ramipril is between about 95% to 100% or between 95% to 100% coated by the blending agent.

Pharmaceutical compositions of the present invention may also include pharmaceutically acceptable additives into any suitable type of unit dosage form. Suitable additives include, but are not limited to, diluents, binders, vehicles, carriers, excipients, binders, disintegrating agents, lubricants, swelling agents, solubilizing agents, wicking agents, cooling agents, preservatives, stabilizers, sweeteners, flavors, polymers etc. While any pharmaceutically acceptable additive is contemplated by the present invention, it should be understood that the additive(s) selected for compounding with coated ramipril particles should not defeat the stability objectives of the present invention. Even though some pharmaceutically acceptable additives may cause ramipril to degrade, such additives may be suitable for the present invention so long as such additives do not cause ramipril, as it is combined with a blending agent, to degrade. Moreover, in certain preferred embodiments, such additives also will not affect the bioavailability of the other active agent formulated in combination with ramipril.

Examples of excipients include, but are not limited to, acacia, alginic acid, croscarmellose, gelatin, gelatin hydrosylate, mannitol, plasdone, sodium starch glycolate, sorbitol, sucrose, and xylitol. For molded or compressed tablet formulations, suitable excipients that may be used include amorphous lactose, beta lactose, microcrystalline cellulose, croscarmellose sodium, dicalcium phosphate, carboxymethyl cellulose, hydroxypropyl cellulose, polyethylene gylcols, sodium lauryl sulfate, and the like.

Examples of additional stabilizers or preservatives include, but are not limited to, parahydroxybenzoic acid alkyl esters, antioxidants, antifungal agents, and other stabilizers/preservatives known in the art.

Examples of coloring agents include, but are not limited to, water soluble dye, Aluminum Lake, ion oxide, natural colors, titanium oxide, and the like.

Examples of diluents or fillers include, but are not limited to, water-soluble and/or water-insoluble tabletting fillers. The water-soluble diluent agent may be constituted from a polyol of less than 13 carbon atoms, in the form of directly compressible material (the mean particle size being between about 100 and about 500 microns or between 100 and 500 microns), in the form of a powder (the mean particle size being less than about 100 microns or less than 100 microns) or a mixture thereof. The polyol is preferably chosen from the group comprising of mannitol, xylitol, sorbitol and maltitol. The water-insoluble diluent agent may be a cellulosic derivative, such as, microcrystalline cellulose or a starch, such as, pregelatinized starch. Especially preferred diluents are those with minimal moisture content, such as lactose monohydrate and magnesium oxide.

Examples of disintegrating agents include, but are not limited to, cross-linked sodium carboxymethylcellulose, crospovidone and their mixtures. A part of the disintegrating agent may be used for the preparation of PPI, cholinergic agonist, parietal activator and/or antacid granules.

Examples of lubricating agents include, but are not limited to, magnesium stearate, stearic acid and its pharmaceutically acceptable alkali metal salts, sodium stearyl fumarate, Macrogol 6000, glyceryl behenate, talc, colloidal silicon dioxide, calcium stearate, sodium stearate, Cab-O-Sil, Syloid, sodium lauryl sulfate, sodium chloride, magnesium lauryl sulfate, talc and their mixtures. A portion of the lubricant may be used as an internal solid lubricant which is blended and granulated with other components of the granulation. Another portion of the lubricant may be added into the final blended material just before compression or encapsulation that coats the outside of the granules in the final blend.

Examples of swelling agents include, but are not limited to, starches; polymers; cellulosic materials, such as, microcrystalline cellulose, hydroxypropylmethyl cellulose, sodium carboxymethylcellulose and ethyl cellulose; waxes such as bees wax; natural materials, such as, gums and gelatins; or mixtures of any of the above.

Examples of polymers include, but are not limited to, polysaccharides, celluloses, and organic moieties such as polyvinyl pyrrolidines and plastics.

Examples of celluloses include, but are not limited to, hydroxypropylcellulose, hydroxypropylmethylcellulose, hydroxylpropyl-methylcellulose, hydroxyethylcellulose, ethylcellulose, cellulose acetate phthalate, cellulose acetate, polyvinyl acetate phthalate, polyvinylpyrrolidone, gelatin, hydroxypropyl methyl cellulose acetate succinate, hydroxypropyl methyl cellulose succinate, hydroxylpropyl cellulose acetate succinate, hydroxyethyl methyl cellulose succinate, hydroxyethyl cellulose acetate succinate, hydroxypropyl methyl cellulose phthalate, hydroxethyl methyl cellulose acetate succinate, hydroxyethyl methyl cellulose acetate phthalate, carboxyethyl cellulose, carboxymethyl cellulose, cellulose acetate phthalate, methyl cellulose acetate phthalate, ethyl cellulose acetate phthalate, hydroxypropyl cellulose acetate phthalate, hydroxypropyl methyl cellulose acetate phthalate, hydroxypropyl cellulose acetate phthalate succinate, hydroxypropyl methylcellulose acetate succinate phthalate, hydroxypropyl methyl cellulose succinate phthalate, cellulose propionate phthalate, hydroxypropyl cellulose butyrate phthalate, cellulose acetate trimellitate, methyl cellulose acetate trimellitate, ethyl cellulose acetate trimellitate, hydroxypropyl cellulose acetate trimellitate, hydroxypropyl methyl cellulose acetate trimellitate, hydroxypropyl cellulose acetate trimelllitate succinate, cellulose propionate trimellitate, cellulose butryrate trimellitate, cellulose acetate terephthalate, cellulose acetate isophthalate, cellulose acetate pyridine dicarboxylate, salicylic acid cellulose acetate, hydroxypropyl salicylic acid cellulose acetate, ethylbenzoic acid cellulose acetate, hydroxypropyl ethylbenzoic acid cellulose acetate, ethyl phthalic acid cellulose acetate, ethyl nicotinic acid, cellulose acetate, ethyl picolinic acid cellulose acetate.

Other polymers that may be suitable for use with the present invention include, but are not limited to, acrylate and methacrylate copolymers. Exemplary commercial grades of such copolymers include the EUDRAGIT® series, which are copolymers of methacrylates, acrylates, carboxylic acid-functionalized vinyl polymers, such as the carboxylic acid functionalized polymethacrylates and carboxylic acid functionalized polyacrylates, amine-functionalized polyacrylates and polymethacrylates; proteins such as gelatin and albumin, and carboxylic acid functionalized starches such as starch glycolate, carboxylic acid functionalized polymethyacrylates, carboxylic acid functionalized polyacrylate, amine-functionalized polyacrylates, amine-functionalized polymethacrylates, proteins, carboxylic acid functionalized starches, vinyl polymers and copolymers having at least one substituent selected from the group consisting of hydroxyl, alkylacyloxy, and cyclicamido; polyvinyl alcohols that have at least a portion of their repeat units in the unhydrolyzed (vinyl acetate) form; polyvinyl alcohol polyvinyl acetate copolymers; polyvinyl pyrrolidone; polyethylene polyvinyl alcohol copolymers, polyoxyethylene-polyoxypropylene copolymers, alkylacyloxy-containing repeat units, or cyclicamido-containing repeat units; polyvinyl alcohols that have at least a portion of their repeat units in the unhydrolyzed form; polyvinyl alcohol polyvinyl acetate copolymers; polyethylene glycol, polyethylene glycol polypropylene glycol copolymers, polyvinyl pyrrolidone polyethylene polyvinyl alcohol copolymers, and polyoxyethylene-polyoxypropylene block copolymers.

The flavouring maybe advantageously chosen to give a combination of fast onset and long-lasting sweet taste and get a “round feeling” in the mouth with different textures or additives. Cooling agents can also be added in order to improve the mouth feeling and provide a synergy with flavours and sweetness. Various other materials may be present as coatings or to otherwise modify the physical form of the dosage unit. For instance, tablets or capsules may be coated with shellac, sugar or both.

Additional illustrations of adjuvants which may be incorporated in the tablets include, but are not limited to, a binder such as gum tragacanth (arabic), acacia, corn starch, potato starch, alginic acid, povidone, acacia, alginic acid, ethylcellulose, methylcellulose, microcrystalline cellulose, a derivatized cellulose, such as carboxymethyl cellulose, sodium carboxymethyl cellulose, hydroxyethyl cellulose, hydroxypropyl methylcellulose, and hydroxypropyl cellulose, dextrin, gelatin, glucose, guar gum, hydrogenated vegetable oil, type I, polyethylene glycol, lactose, lactose monohydrate, compressible sugars, sorbitol, mannitol, dicalcium phosphate dihydrate, tricalcium phosphate, calcium sulfate dihydrate, maltodextrins, lactitol, magnesium carbonate, xylitol, magnesium aluminum silicate, maltodextrin, methylcellulose, hydroxypropylcellulose, polyethylene, polyethylene oxide, polymethacrylates, plasdone, sodium alginate, starch, pregelatinized starch, zein or the like; a sweetening agent such as sucrose, potassium acesulfame, aspartame, lactose, dihydrochalcone neohesperidine, saccharin, sucralose, polyols such as xylitol, mannitol, and maltitol, sodium saccharide, Asulfame-K, Neotame®, glycyrrhizin, malt syrup and combinations thereof; a flavoring such as berry, orange, peppermint, oil of wintergreen, cherry, citric acid, tartaric acid, menthol, lemon oil, citrus flavor, common salt, and other flavors known in the art.

Pharmaceutical compositions of the present invention can be administered orally or internally to subjects. This can be accomplished, for example, by administering to the subject a solid or liquid oral dosage form by mouth or via a gastric feeding tube, a duodenal feeding tube, a nasogastric (ng) tube, a gastrostomy, or other indwelling tubes placed in the GI tract. Other forms of the drug may be in suppositories, suspensions, liquids, powders, creams, transdermal patches, and depots.

Oral pharmaceutical compositions of the present invention are generally in the form of individualized or multi unit doses, such as tablets, caplets, powders, suspension tablets, chewable tablets, rapid melt tablets, capsules, e.g., a single or double shell gelatin capsule, tablet-filled capsules, effervescent powders, effervescent tablets, pellets, granules, liquids, solutions, or suspensions, respectively.

While the present invention contemplates any solid dosage form suitable for oral administration, ramipril tablets, capsules, tablet-filled capsules and caplets are especially preferred. When the pharmaceutical compositions of the present invention are formed into tablets or caplets, it is to be understood that the tablets or caplets may be scored, and that they may be of any suitable shape and size, such as round, square, rectangular, oval, diamond, pentagon, hexagon or triangular, so long as the objectives of the present invention are not defeated. It is to be further understood that when tablet-filled capsules are selected, the tablets utilized therewith may be formed into shapes that either (a) correspond to the capsules to permit over-coating or encapsulation via the capsules or (b) readily fit inside the capsules.

The oral pharmaceutical compositions may contain a drug in any therapeutically effective amount, such as from about 0.001 mg or from 0.001 mg or less to about 200 mg or less than 200 mg or more, or preferably from about 0.01 mg to about 100 mg or from 0.01 mg to 100 mg or preferably from about 0.1 mg to about 50 mg or from 0.1 mg to 50 mg. Preferably, the dosage range will be between about 1.25 mg to about 25 mg per patient per day or 1.25 mg to 25 mg per patient per day; more preferably about 10 mg to about 20 mg per patient per day or 10 mg to 20 mg per patient per day, and most preferably about 10 mg or 20 mg per day or 10 mg or 20 mg per day.

By way of example, a particularly preferred stabilized oral unit dose or composition of the present invention may contain ramipril in a dosage amount of about 1.25 mg, about 2.5 mg, about 5 mg, about 7.5 mg, about 10 mg, 12.5 mg, about 15 mg, about 20 mg, about 25 mg, about 30 mg, about 40 mg, about 50 mg, about 60 mg, about 70 mg, about 75 mg, about 80 mg, about 90 mg, or about 100 mg or 1.25 mg, 2.5 mg, 5 mg, 7.5 mg, 10 mg, 12.5 mg, 15 mg, 20 mg, 25 mg, 30 mg, 40 mg, 50 mg, 60 mg, 70 mg, 75 mg, 80 mg, 90 mg, or 100 mg. Of course, it should be appreciated that a particular unit dosage form and amount can be selected to accommodate the desired frequency of administration used to achieve a specified daily dosage and therapeutic effect.

Of particular interest are oral dosage forms that comprise stabilized ramipril having 1.25, 2.5, 5, 10, 15 and 20 mg ramipril per unit dosage form. Such dosage forms can be tablets, caplets, capsules or tablet-filled capsules.

Of particular interest are oral dosage forms that comprise stabilized ramipril and chlorthalidone having 6.5, 12.5, and 25 mg chlorthalidone per unit dosage form.

Of particular interest are oral dosage forms that comprise stabilized ramipril and hydrochlorothiazide having 6.5 and 25 mg hydrochlorothiazide per unit dosage form.

Consistent with the present invention, the dosage forms of the instant invention may be administered to individuals on a regimen of one, two or more doses per day, at any time of the day.

The dosage of active ingredient in the compositions of the invention may be varied; however, it is necessary that the amount of the active ingredient be such that a suitable dosage form is obtained. The active ingredient may be administered to patients (animals and human) in need of such treatment in dosages that will provide optimal pharmaceutical efficacy. The selected dosage depends upon the desired therapeutic effect, on the route of administration, and on the duration of the treatment. The dose will vary from patient to patient depending upon the nature and severity of disease, the patient's weight, special diets being followed by a patient, concurrent medication, and other factors, recognized by those skilled in the art. Based upon the foregoing, precise dosages depend on the condition of the patient and are determined by discretion of a skilled clinician. Generally, ramipril daily dosage levels of between about 0.010 to about 1.5 mg/kg or 0.010 to 1.5 mg/kg of body weight are administered daily to mammalian patients, e.g., humans having a body weight of about 70 kg. The ramipril dosage range will generally be about 1.25 mg to 50 mg or 1.25 mg to 50 mg per patient per day, administered in single or multiple doses.

Nonetheless, it should be understood that safe and effective amounts of ramipril and the other active agents utilized in accordance with the present invention will vary with the particular cardiovascular disorder, conditions and/or symptoms being treated, the age, weight and physical conditions of the subjects being treated, the severity of the cardiovascular disorder, conditions and/or symptoms, the duration of treatments, the nature of concurrent therapies, the specific dosage form employed, the particular pharmaceutically acceptable carriers utilized, and like factors within the knowledge and expertise of the attending physicians. Exemplary safe and effective amounts of ramipril include those amounts mentioned herein, administered one or more times per day, as will be more fully describe herein below. Exemplary safe and effective amounts of other active agents such as chlorthalidone or hydrochlorothiazide and other diuretics, or of calcium channel blockers and beta blockers are readily apparent to those skilled in the art and can be found in sources such as the Physician's Desk Reference, 59^{th} Edition (2005).

The present invention is also generally directed towards methods of making pharmaceutical compositions with improved stability, bioavailability and shelf-life. The following methods of making a pharmaceutical compositions in accordance with the present can be used with any drug. Specifically, the methods of the present invention are directed to making pharmaceutical compositions comprising any drug that is susceptible to degradation when exposed to the environment or exposed to physical stresses during the manufacturing process.

The pharmaceutical compositions of the present invention can be made by first combining a drug with a blending agent so that the drug is coated with a blending agent before being processed into tablets. Combining the drug with the blending agent can be accomplished by blending, mixing, milling or co-milling, compressing, granulating, suspending, dissolving or precipitating the drug and the blending agent together.

Preferably, the combined drug and blending agent is suitable for use in preparing dosage forms by processes including, but not limited to, dry blend, direct compression formulations and hot melt extrusion processes.

Preferably, the methods of the present invention comprise an ACE inhibitor and more preferably, ramipril.

Methods of the present invention comprise combining ramipril with a blending agent. Such methods can also further comprise adding an additive such as, but not limited to, a polymer, diluent, disintigrant or a combination thereof, before or after the ramipril is combined with the blending agent. Combining ramipril with the blending agent can be accomplished by blending, mixing, milling or co-milling, compressing, granulating, suspending, dissolving or precipitating the drug and the blending agent together.

In various embodiments, the invention contemplates methods comprising combining a blending agent and ramipril before the ramipril is further processed with at least one other active agent and other excipients into a dosage form. Preferably, the blending agent and ramipril are pre-blended or co-milled before the ramipril is further processed into the formulations of the instant invention. The invention also contemplates methods that further comprise adding additives including, but not limited to, diluents, binders, vehicles, carriers, excipients, binders, disintegrating agents, lubricants, swelling agents, solubilizing agents, wicking agents, cooling agents, preservatives, stabilizers, sweeteners, flavors, polymers, to the pre-blended or co-milled ramipril and blending agent.

In preferred embodiments methods of the present invention comprise first pre-blending and or co-milling ramipril with a blending agent. Such methods can also further include additional steps comprising combining the pre-blended ramipril and blending agent along with a polymer, diluent, disintigrant or a combination thereof. Further, the methods will comprise the additional step of adding at least one other active agent to the co-milled or pre-blended ramipril. In alternate embodiments, the methods comprise co-milling and/or pre-blending ramipril and at least one other active agent with the blending agent prior to further formulation of ramipril and the other active agent into a dosage form.

In other preferred embodiments the methods of the present invention comprise pre-blending ramipril with a blending agent and then co-milling the ramipril and the blending agent. Such methods can also further include additional steps comprising combining the co-milled ramipril and blending agent along with a polymer, diluent, disintigrant or a combination thereof. Further, the methods will comprise the additional step of adding at least one other active agent to the co-milled or pre-blended ramipril. In alternate embodiments, the methods comprise co-milling or pre-blending ramipril and at least one other active agent with the blending agent prior to further formulation of ramipril and the other active agent into a dosage form.

In other embodiments the methods of the present invention comprises blending ramipril with a blending agent; co-milling the ramipril and the blending agent and then re-blending the ramipril with the blending agent. Such methods can also further include additional steps comprising combining the ramipril and blending agent along with a polymer, diluent, disintigrant or a combination thereof. Further, the methods will comprise the additional step of adding at least one other active agent to the co-milled or pre-blended ramipril. In alternate embodiments, the methods comprise co-milling or pre-blending ramipril and at least one other active agent with the blending agent prior to further formulation of ramipril and the other active agent into a dosage form.

In yet other embodiments, the method of the present invention comprises blending ramipril with a polymer and co-milling the ramipril and polymer with a blending agent. Such methods can also further include additional steps comprising combining the ramipril with a second polymer, diluent, disintigrant or a combination thereof, before or after being co-milled with the blending agent. Further, the methods will comprise the additional step of adding at least one other active agent to the co-milled or pre-blended ramipril. In alternate embodiments, the methods comprise co-milling or pre-blending ramipril and at least one other active agent with the blending agent prior to further formulation of ramipril and the other active agent into a dosage form.

In one embodiment the method of making solid oral ramipril pharmaceutical compositions comprises blending coated ramipril with a blending agent; co-milling the coated ramipril and the blending agent; and re-blending the coated ramipril with a blending agent. Additionally, a polymer, a diluent, a lubricant or a disintigrant can be combined with the ramipril before or after being milled. Further, the methods will comprise the additional step of adding at least one other active agent to the co-milled or pre-blended ramipril. In alternate embodiments, the methods comprise co-milling or pre-blending ramipril and at least one other active agent with the blending agent prior to further formulation of ramipril and the other active agent into a dosage form.

In the above methods, one purpose of the pre-blending and co-milling the blending agent and ramipril before the ramipril is further processed into tablets is to facilitate coating the ramipril with the blending agent. In all of the above methods the blending agent coats the ramipril. Preferably the blending agent coats between about 50% to 100% of the ramipril, or between about 75% to 100% or 50% to 100% of the ramipril, or between about 85% to about 100% or 85% to 100% and most preferably between about 95% to 100% or 95% to 100%. Also in all of the above methods the preferred blending agent is glyceryl behenate

In a particularly preferred embodiment, ramipril and glyceryl behenate are first co-milled, then followed by additional steps wherein, sodium stearyl fumarate and croscarmellose sodium are added to the ramipril and glyceryl behenate blend. Further, the methods will comprise the additional step of adding at least one other active agent to the co-milled or pre-blended ramipril. In alternate embodiments, the methods comprise co-milling or pre-blending ramipril and at least one other active agent with the blending agent prior to further formulation of ramipril and the other active agent into a dosage form.

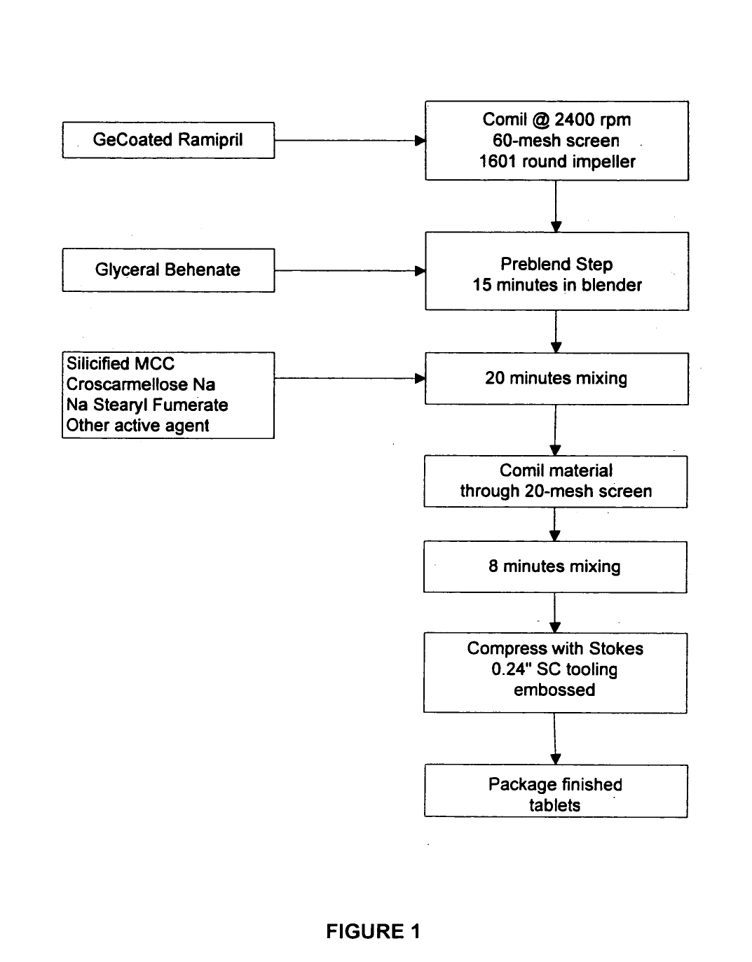

FIG. 1 shows one method of making pharmaceuticals of the present invention comprising GECoated ramipril. GEcoated ramipril is pre-milled though a 60-mesh screen. The milled ramipril is then pre-blended with glyceryl behenate for 15 minutes in a blender that has been grounded to reduce electrostatic charges. Croscarmellose sodium, sodium stearyl fumerate and silicified microcrystalline cellulose are added to the mixture and mixed for another 20 minutes. The co-milled mixture is then passed through a 20-mesh sieve. The sieved mixture is then placed into blender and mixed for an additional 8 minutes. The mixture is then compressed with a tablet press. The tablets finished tablets then can be packaged.

This process can be scaled, for example, to about 6 kg, in a 16-quart V-shell PK blender, and larger as needed. Tablets can be produced with a Fette P1200 24-station press, or similar equipment.

In the alternative, pharmaceutical compositions made by the above process can be formulated with uncoated ramipril as well. Also, microcrystalline cellulose can be replaced with diluents and fillers including but not limited to Ceolus®, lactose, anhydrous lactose, lactose monohydrate, starch, spray-dried mannitol (Pearlitol 200 SD), Prosolv® SMCC 90, or a combination thereof. Also, glyceryl behenate can be replaced with magnesium stearate.

The method, as shown in FIG. 1, can be used with any type of ramipril. Also, the mixing times and other parameters of the process can be varied to achieve the pharmaceutical compositions of the present invention comprising ramipril, wherein the ramipril has a low rate of degradation compared to current formulations.

An article of manufacture, as contemplated by the present invention, comprises a container holding a pharmaceutical composition suitable for oral administration of stabilized ramipril in combination with at least one other active agent along with printed labeling instructions providing a discussion of when a particular dosage form should be administered.

The composition will be contained in any suitable container capable of holding and dispensing the dosage form and which will not significantly interact with the composition and will further be in physical relation with the appropriate labeling advising that a dosage form is more stable and/or bioavailable with extended shelf life.

The labeling instructions will be consistent with the methods of treatment as described hereinbefore. The labeling may be associated with the container by any means that maintain a physical proximity of the two, by way of non-limiting example, they may both be contained in a packaging material such as a box or plastic shrink wrap or may be associated with the instructions being bonded to the container such as with glue that does not obscure the labeling instructions or other bonding or holding means.

The compositions, of the present invention, comprising ramipril in combination with at least one other active agent, can be administered to a subject for the treatment of cardiovascular disorders. Cardiovascular disorders include but are not limited to, hypertension, heart failure, congestive heart failure, myocardial infarction, atherosclerotic cardiovascular disease, asymptomatic left ventricular dysfunction, chronic renal insufficiency, and diabetic or hypertensive nephropathy.

An embodiment of the subject invention is a pharmaceutical composition comprising ramipril, another active agent, and a blending agent, wherein the ramipril is coated by the blending agent, and wherein the blending agent is selected from glyceryl behenate, glyceryl stearate, stearyl alcohol, macrogol stearate ether, palmitostearate, ethylene glycol, polyethylene glycol, stearic acid, cetyl alcohol, lauryl alcohol, amylopectin, poloxymer or combinations thereof.

In another embodiment of the subject invention, the rate of decomposition of the ramipril to ramipril-diketopiperazine is less than about 0.3% by weight during about the first three months.

In another embodiment of the subject invention, the rate of decomposition of the ramipril to ramipril-diketopiperazine is less than about 3.0% by weight during about the first thirty-six months.

In another embodiment of the subject invention, the rate of decomposition of the ramipril to ramipril-diketopiperazine is less than about 0.11% by weight, on average, per month.

Another embodiment of the subject invention is a pharmaceutical composition comprising ramipril, another active agent, and a blending agent, wherein the ramipril is coated by a blending agent and wherein the rate of decomposition of the ramipril to ramipril-diketopiperazine is less than about 0.4% of the total weight of ramipril during the first 3 months when the pharmaceutical composition is at room temperature.

In another embodiment of the subject invention, the rate of decomposition is about 0.3%.

Another embodiment of the subject invention is a pharmaceutical composition comprising ramipril, another active agent, and a blending agent, wherein the ramipril is coated by a blending agent and wherein the rate of decomposition of the ramipril to ramipril-diketopiperazine is less than about 1.0% of the total weight of ramipril during the first 6 months when the pharmaceutical composition is at room temperature.

In another embodiment of the subject invention, the rate of decomposition is about 0.75%.

Another embodiment of the subject invention is a pharmaceutical composition comprising ramipril, another active agent, and a blending agent, wherein the ramipril is coated by a blending agent and wherein the rate of decomposition of the ramipril to ramipril-diketopiperazine is less than about 3.0% of the total weight of ramipril during the first 36 months when the pharmaceutical composition is at room temperature.

In another embodiment of the subject invention, the rate of decomposition is about 2.0%.

In another embodiment of the subject invention, the rate of decomposition is about 1.5%.

Another embodiment of the subject invention is a pharmaceutical composition comprising ramipril, another active agent, and a blending agent, wherein the ramipril is coated by a blending agent and wherein the rate of decomposition of the ramipril to ramipril-diketopiperazine is less than about 0.15%, on average, of the total weight of ramipril per month when the pharmaceutical compositions are at room temperature.

In another embodiment of the subject invention, the rate of decomposition is about 0.09% or less.

In another embodiment of the subject invention, the blending agent is glyceryl behenate.

In another embodiment of the subject invention, about 50 to 100% of the ramipril is coated by the blending agent.

In another embodiment of the subject invention, about 75 to 100% of the ramipril is coated by the blending agent.

In another embodiment of the subject invention, about 95 to 100% of the ramipril is coated by the blending agent.

In another embodiment of the subject invention, the blending agent is at least 0.1% by weight.

In another embodiment of the subject invention, the blending agent is at least 1% by weight.

In another embodiment of the subject invention, the blending agent is at least 4% by weight.

In another embodiment of the subject invention, the ramipril is substantially stable against decomposition into a degradant product.

In another embodiment of the subject invention, the degradant product is ramipril-diacid or ramipril-diketopiperazine.

In another embodiment of the subject invention, the ramipril is coated ramipril.

In another embodiment of the subject invention, the composition is a solid dosage form.

In another embodiment of the subject invention, the composition is an oral dosage form.

In another embodiment of the subject invention, the composition is a tablet, caplet or capsule.

In another embodiment of the subject invention, the composition is a tablet.

In another embodiment of the subject invention, the composition further comprises an excipient.

In another embodiment of the subject invention, the ramipril is in the amount of about 0.1 mg to 50 mg.

In another embodiment of the subject invention, the ramipril is in the amount of about 1 mg to 30 mg.

In another embodiment of the subject invention, the ramipril is in the amount of about 2.5 mg.

In another embodiment of the subject invention, the ramipril is in the amount of about 5 mg.

In another embodiment of the subject invention, the ramipril is in the amount of about 10 mg.

In another embodiment of the subject invention, the ramipril is in the amount of about 15 mg.

In another embodiment of the subject invention, the ramipril is in the amount of about 20 mg.

In another embodiment of the subject invention, the active agent is a diuretic, a angiotensin receptor blocker, an ACE inhibitor, a cholesterol lowering drug, a calcium channel blocker, a beta blocker, a glucose lowering agent, or oral hypoglycemics.

In another embodiment of the subject invention, the active agent is a diuretic.

In another embodiment of the subject invention, the diuretic is chlorthalidone.

In another embodiment of the subject invention, the chlorthalidone is in the amount of about 0.1 mg to 50 mg.

In another embodiment of the subject invention, the chlorthalidone is in the amount of about 1 mg to 30 mg.

In another embodiment of the subject invention, the chlorthalidone is in the amount of about 6.5 mg.

In another embodiment of the subject invention, the chlorthalidone is in the amount of about 12.5 mg.

In another embodiment of the subject invention, the chlorthalidone is in the amount of about 25 mg.

In another embodiment of the subject invention, the diuretic is hydrochlorothiazide.

In another embodiment of the subject invention, the hydrochlorothiazide is in the amount of about 0.1 mg to 50 mg.

In another embodiment of the subject invention, the hydrochlorothiazide is in the amount of about 1 mg to 30 mg.

In another embodiment of the subject invention, the hydrochlorothiazide is in the amount of about 6.5 mg.

In another embodiment of the subject invention, the hydrochlorothiazide is in the amount of about 25 mg.

Another embodiment of the subject invention is a method of making a pharmaceutical composition comprising coating ramipril with a blending agent, wherein the pharmaceutical composition further comprises another active agent.

Another embodiment of the subject invention is a method of making a pharmaceutical composition comprising:

a) pre-blending milled ramipril with a blending agent; and

b) combining the product of step a) with another active agent, wherein the blending agent is glyceryl behenate, glyceryl stearate, stearyl alcohol, macrogol stearate ether, palmitostearate, ethylene glycol, polyethylene glycol, stearic acid, cetyl alcohol, lauryl alcohol, amylopectin, poloxymer or combinations thereof.

Another embodiment of the subject invention is a method of making a pharmaceutical composition comprising:

a) pre-blending and co-milling ramipril with a blending agent; and

b) combining the product of step a) with another active agent,

wherein the blending agent is glyceryl behenate, glyceryl stearate, stearyl alcohol, macrogol stearate ether, palmitostearate, ethylene glycol, polyethylene glycol, stearic acid, cetyl alcohol, lauryl alcohol, amylopectin, poloxymer or combinations thereof.

In another embodiment of the subject invention, the method of making a pharmaceutical composition further comprising blending the ramipril with the blending agent before the ramipril and blending agent are co-milled.

In another embodiment of the subject invention, the method of making a pharmaceutical composition further comprising blending the ramipril with the blending agent after the ramipril and blending agent are co-milled.

In another embodiment of the subject invention, the method of making a pharmaceutical composition further comprising adding a diluent, lubricant, disintegrant or a combination thereof.

In another embodiment of the subject invention, the method of making a pharmaceutical composition further comprising compressing the product of step b) into tablets.

In another embodiment of the subject invention, the blending agent is glyceryl behenate.

In another embodiment of the subject invention, the blending agent is at least 0.1% by weight.

In another embodiment of the subject invention, the blending agent is at least 1% by weight.

In another embodiment of the subject invention, the blending agent is at least 4% by weight.

In another embodiment of the subject invention, the ramipril is coated ramipril.

In another embodiment of the subject invention, the composition is a solid dosage form.

In another embodiment of the subject invention, the composition is an oral dosage form.

In another embodiment of the subject invention, the composition is a tablet, caplet or capsule.

In another embodiment of the subject invention, the composition is a tablet.

In another embodiment of the subject invention, the ramipril is in the amount of about 0.1 mg to 50 mg.

In another embodiment of the subject invention, the ramipril is in the amount of about 1 mg to 30 mg.

In another embodiment of the subject invention, the ramipril is in the amount of about 2.5 mg.

In another embodiment of the subject invention, the ramipril is in the amount of about 5 mg.

In another embodiment of the subject invention, the ramipril is in the amount of about 10 mg.

In another embodiment of the subject invention, the ramipril is in the amount of about 15 mg.

In another embodiment of the subject invention, the ramipril is in the amount of about 20 mg.

In another embodiment of the subject invention, the active agent is a diuretic, a angiotensin receptor blocker, an ACE inhibitor, a cholesterol lowering drug, a calcium channel blocker, a beta blocker, a glucose lowering agent, or oral hypoglycemics.

In another embodiment of the subject invention, the active agent is a diuretic.

In another embodiment of the subject invention, the diuretic is chlorthalidone.

In another embodiment of the subject invention, the chlorthalidone is in the amount of about 0.1 mg to 50 mg.

In another embodiment of the subject invention, the chlorthalidone is in the amount of about 1 mg to 30 mg.

In another embodiment of the subject invention, the chlorthalidone is in the amount of about 6.25 mg.

In another embodiment of the subject invention, the chlorthalidone is in the amount of about 12.5 mg.

In another embodiment of the subject invention, the chlorthalidone is in the amount of about 25 mg.

In another embodiment of the subject invention, the diuretic is hydrochlorothiazide.

In another embodiment of the subject invention, the hydrochlorothiazide is in the amount of about 0.1 mg to 50 mg.

In another embodiment of the subject invention, the hydrochlorothiazide is in the amount of about 1 mg to 30 mg.

In another embodiment of the subject invention, the hydrochlorothiazide is in the amount of about 6.5 mg.

In another embodiment of the subject invention, the hydrochlorothiazide is in the amount of about 25 mg.

In another embodiment of the subject invention, the method of making a pharmaceutical composition further comprising combining the produce of step b) with microcrystalline cellulose and croscarmellose sodium.

Another embodiment of the subject invention is a product made by the above methods.

Another embodiment of the subject invention is a method of treating a cardiovascular disorders in a human comprising administering to the human an effective amount of any of the above compositions.

In another embodiment of the subject invention, the cardiovascular disorder is hypertension, heart failure, congestive heart failure, myocardial infarction, atherosclerotic cardiovascular disease, asymptomatic left ventricular dysfunction, chronic renal insufficiency, and diabetic or hypertensive nephropathy.

As shown in the examples below, the introduction of another active agent into the ramipril formulation of the present invention did not result in a significant change in the long term stability of ramipril in the formulation. This result was unexpected given the known stability problems associated with prior ramipril formulations. Moreover, it was also surprising that the diuretics formulated in combination with ramipril demonstrate the same stability or bioavailability as the current single agent formulations.

The examples throughout herein and that follow are provided solely to illustrate representative embodiments of the invention. Accordingly, it should be understood, that the invention is not to be limited to the specific conditions or details described in these or any other example discussed herein, and that such examples are not to be construed as limiting the scope of the invention in any way. Throughout the specification, any and all references are specifically incorporated herein by reference in their entireties.

### EXAMPLES

### Example 1

### Methods of Making Combinations

The ramipril/chlorthalidone combination tablets were made by pre-blending the coated ramipril with glyceryl behenate, sodium stearyl fumarate and croscarmellose sodium in a 16-quart V-shell blender and blending for a suitable mount of time, then mill-blending the mixture through a Quadro Co-mil. Chlorthalidone was then added to the mixture with microcrystalline cellulose, sodium stearyl fumarate and croscarmellose sodium in a 16-quart container and mixed, then compressed on a Stokes B2 tablet press, tooled with 16 stations with ¼″ standard concave (about 100 mg tablet weight) or 5/16″ standard concave (about 200 mg tablet weight) double-sided debossed tooling at about 48 rpm.

Ramipril/hydrochlorthiazide combination tablets were made by pre-milling coated ramipril (ramipril coated with hydroxypropyl methylcellulose) through a 40 or 60 mesh screens and then pre-blended with a blending agent such as, glyceryl behenate. Hydrochlorthiazide, silicified microcrystalline cellulose and croscarmellose sodium were added and mixed for an additional period of time. The mixture was co-milled through a 20 mesh screen and blended. The mixture was compressed into tablets.

### Example 2

### Combination of Ramipril and Chlorthalidone

This study was conducted as a single-dose, randomized, open-label, three-way crossover design in healthy male and female volunteers. Forty-five subjects (40%-60% female) were enrolled in the study. Following a 14-day screening period, subjects underwent a two-stage randomization process for treatment group and sequence. The following treatments were utilized.

| | | Treatment | | |
|---|---|---|---|---|
| Treatment | | Ramipril- | Chlorthalidone Commercial Tablet | Chlorthalidone |
| Group | N | Chlorthalidone Tablet | (chlorthalidone, USP) | Tablet |
| I | 15 | 4 × 2.5 mg/6.25 mg | 1 × 25 mg (B) | 4 × 6.25 mg (C) |
| | | (A) | | |
| II | 15 | 2 × 10 mg/12.5 mg | 1 × 25 mg (E) | 2 × 12.5 mg (F) |
| | | (D) | | |
| III | 15 | 1 × 20 mg/25 mg (G) | 1 × 25 mg (H) | 1 × 25 mg (I) |

Subjects were randomized to Treatment Group I, II, or III and received three treatments (ramipril-chlorthalidone tablet, chlorthalidone commercial tablet, and chlorthalidone tablet) in random order. Treatments were separated by a 3-week washout period.

During each period, on the evening before dosing, subjects were admitted to the clinical research unit (CRU) and underwent a supervised overnight fast for at least 10 hours before dosing. Study drug was administered with 240 mL (8 fluid ounces) of water.

Serial blood sampling was performed at specified times pre- and post dose for quantitation of chlorthalidone in whole blood. Vital signs, 12-lead electrocardiogram (ECGs), clinical laboratory determinations, and adverse event (AE) assessments were done at specified times (Table 1). A final safety assessment was performed at the end of the last period. During each period, subjects were confined to the CRU for approximately 60 hours (3 nights and 2 days). The estimated time of participation in the study including Screening was 9 weeks.

The single-dose treatments administered in this study are listed in Table 2.

TABLE 2
Treatments Administered

| Treat- | | | | |
|---|---|---|---|---|
| ment | | | Dose (mg) | |
| Group | | Treatment | Ramipril | Chlorthalidone |
| I | A: | 4 × 2.5 mg/6.25 mg ramipril- | 10 | 25 |
| | | chlorthalidone tablets | | |
| | B: | 1 × 25 mg chlorthalidone | 0 | 25 |
| | | commercial tablet | | |
| | C: | 4 × 6.25 mg chlorthalidone | 0 | 25 |
| | | tablets | | |
| II | D: | 2 × 10 mg/12.5 mg ramipril- | 20 | 25 |
| | | chlorthalidone tablets | | |
| | E: | 1 × 25 mg chlorthalidone | 0 | 25 |
| | | commercial tablet | | |
| | F: | 2 × 12.5 mg chlorthalidone | 0 | 25 |
| | | tablets | | |
| III | G: | 1 × 20 mg/25 mg ramipril- | 20 | 25 |
| | | chlorthalidone tablet | | |
| | H: | 1 × 25 mg chlorthalidone | 0 | 25 |
| | | commercial tablet | | |
| | I: | 1 × 25 mg chlorthalidone | 0 | 25 |
| | | tablet | | |

Subjects were instructed to swallow the treatment whole and not to chew or crush it. The test products were administered as a single oral dose taken with 240 mL water.

There were 3 kinds of test products used in this study:
Ramipril/chlorthalidone combination tablets manufactured by King Pharmaceuticals, Inc.: 2.5 mg/6.25 mg, 10 mg/12.5 mg, and 20 mg/25 mg.
Chlorthalidone commercial 25 mg tablets (chlorthalidone, USP,) manufactured by Mylan Pharmaceuticals, Inc.
Chlorthalidone tablets manufactured by King Pharmaceuticals, Inc.: 6.25 mg, 12.5 mg, and 25 mg.

The following noncompartmental pharmacokinetic parameters for chlorthalidone were calculated from the whole blood concentrations using WinNonlin Pro Version 4. Actual sample times were used in the calculations. Whole blood concentrations below the limit of quantitation (BLQ) prior to the first quantifiable concentrations were replaced with zero for the calculation of the pharmacokinetic parameters. Concentrations BLQ after the first quantifiable concentration were treated as missing. In this study, there were no BLQ values after the first quantifiable concentration; therefore, any values listed as missing in the concentration tables are true missing values.
AUC₀₋ₜ The area under the whole blood concentration versus time curve, from time 0 to time t, where Cₜ is the last quantifiable concentration, as calculated by the linear trapezoidal method.
AUC_{0-inf} The area under the whole blood concentration versus time curve, from time 0 to infinity, calculated as AUC₀₋ₜ+Cₜ/Kₑₗ, where Kₑₗ is the terminal elimination rate constant.
Cₘₐₓ Maximum (peak) observed whole blood concentration.
Tₘₐₓ Time of the maximum (peak) observed whole blood concentration.
Kₑₗ Apparent terminal elimination rate constant, calculated from the linear regression of the terminal linear portion of the ln(concentration) versus time curve, where Kₑₗ is the absolute value of the slope.
T_{1/2} Apparent terminal elimination half-life, calculated as ln(2)/Kₑₗ.

Linear regressions were performed using at least three data points. Kₑₗ was assigned if the terminal phase was apparent, if Tmax was not one of the 3 last data points, and if the R² value (correlation of linear regression) was greater than 0.8.

Venous blood samples (5 mL) for the Pharmacokinetic studies were obtained from an indwelling catheter (heparin flush as needed) or by direct venipuncture. Blood samples were collected at predose (time 0) and at 0.5, 1, 1.5, 2, 3, 4, 6, 8, 10, 12, 16, 24, 36, 48, 72, 96, and 120 hours postdose.

Whole blood concentrations of chlorthalidone were determined using a specific liquid chromatography/mass spectrometry/mass spectrometry (LC/MS/MS) method. The assay was validated with respect to accuracy, precision, linearity, sensitivity, and specificity.

Data from all subjects with an evaluable profile were included in the pharmacokinetic analysis. Of the 45 subjects enrolled in the study, only 2 subjects did not complete all 3 periods. Data from these 2 subjects were included in the pharmacokinetic summary statistics and in the statistical analysis of the treatment comparisons.

Of the 45 subjects enrolled in this study, 23 were female and 22 were male. Regarding race, 37 subjects were Caucasian, 4 subjects were Hispanic, and 1 subject each was Asian, Black, European/Middle Eastern, and of “other” race. The mean age for all subjects was 25.8 years (range 19-48 years), the mean weight was 71.4 kilograms (range 55.8-94.3 kilograms), and the mean height was 174.3 centimeters (range 160.0-193.0 centimeters). Regarding body frame size, 5 subjects had a small frame, 33 subjects had a medium frame, and 7 subjects had a large frame.

Descriptive statistics including arithmetic mean, standard deviation (SD), coefficient of variation (CV %), standard error of the mean (SEM), number (N), minimum, maximum, and median were presented for whole blood chlorthalidone concentrations at each time point, and for all PK parameters by treatment. Additionally, geometric mean was presented for Cₘₐₓ, AUC₀₋ₜ, and AUC_{0-inf}; and harmonic mean was presented for T_{1/2}.

To evaluate the chlorthalidone bioavailability, analyses of variance (ANOVA) were performed on the ln-transformed Cₘₐₓ, AUC₀₋ₜ, and AUC_{0-inf}. The ANOVA model included terms for treatment, period, sequence, and subject within sequence. A separate ANOVA model was applied to the data from each of the three treatment groups. The bioavailability of chlorthalidone from the ramipril-chlorthalidone tablets was compared to that of the chlorthalidone commercial tablet, and to that of the chlorthalidone tablets. In addition, the bioavailability of chlorthalidone from the chlorthalidone tablets was compared to that of the chlorthalidone commercial tablet. For each comparison, the ratios of least-squares means (LSM) and the 90% confidence intervals (CI) were expressed as a percentage relative to the commercial chlorthalidone tablet, or chlorthalidone tablet. A conclusion of equivalent bioavailability was based on the mean ratios and whether the 90% CI for the ln-transformed Cₘₐₓ, AUC₀₋ₜ, and AUC_{0-inf} were within the 80-125% range where the rate and extent of exposure to chlorthalidone are considered equivalent between the treatments.

The Group I statistical comparisons (% mean ratios and 90% CI) of the chlorthalidone ln-transformed Cₘₐₓ, AUC₀₋ₜ, and AUC_{0-inf} for the 4×2.5 mg/6.25 mg ramipril-chlorthalidone tablets (Treatment A), the 1×25 mg commercial chlorthalidone tablet (Treatment B), and the 4×6.25 mg chlorthalidone tablets (Treatment C) are presented in the following table.

TABLE 4
Relative Bioavailability Results for Whole Blood Chlorthalidone for
Group I (Treatments A, B, and C)

| | Treatment A vs. | Treatment A vs. | Treatment C vs. |
|---|---|---|---|
| Parameter | Treatment B | Treatment C | Treatment B |
| Cₘₐₓ | 95.9% (91.21-100.88%) | 95.0% (90.40-99.92%) | 100.9% (96.00-106.12%) |
| (ng/mL) | | | |
| AUC₀₋ₜ | 99.0% (95.12-103.09%) | 95.8% (92.00-99.66%) | 103.4% (99.36-107.63%) |
| (ng · h/mL) | | | |
| AUC_{0-inf} | 101.5% (97.61-105.48%) | 96.2% (92.59-100.00%) | 105.5% (101.47-109.60%) |
| (ng · h/mL) | | | |

Treatment A = 4 × 2.5 mg/6.25 mg Ramipril-Chlorthalidone Tablets
Treatment B = 1 × 25 mg Commercial Chlorthalidone Tablet
Treatment C = 4 × 6.25 mg Chlorthalidone Tablets

The Group II statistical comparisons (% mean ratios and 90% CI) of the chlorthalidone ln-transformed Cₘₐₓ, AUC0-t, and AUC0-inf for the 2×10 mg/12.5 mg ramipril-chlorthalidone tablets (Treatment D), the 1×25 mg commercial chlorthalidone tablet (Treatment E), and the 2×12.5 mg chlorthalidone tablets (Treatment F) are presented in the following table.

TABLE 5
Relative Bioavailability Results for Whole Blood Chlorthalidone for
Group II (Treatments D, E, and F)

| | Treatment D vs. | Treatment D vs. | Treatment F vs. |
|---|---|---|---|
| Parameter | Treatment E | Treatment F | Treatment E |
| Cₘₐₓ | 94.2% (88.07-100.83%) | 96.0% (89.70-102.69%) | 98.2% (91.77-105.06%) |
| (ng/mL) | | | |
| AUC₀₋ₜ | 96.2% (90.80-101.98%) | 97.3% (91.79-103.09%) | 98.9% (93.35-104.84%) |
| (ng · h/mL) | | | |
| AUC_{0-inf} | 96.4% (91.07-102.10%) | 97.8% (92.35-103.54%) | 98.6% (93.13-104.41%) |
| (ng · h/mL) | | | |

Treatment D = 2 × 10 mg/12.5 mg Ramipril-Chlorthalidone Tablets
Treatment E = 1 × 25 mg Commercial Chlorthalidone Tablet
Treatment F = 2 × 12.5 mg Chlorthalidone Tablets

The Group III statistical comparisons (% mean ratios and 90% CI) of the chlorthalidone ln-transformed Cₘₐₓ, AUC₀₋ₜ, and AUC_{0-inf} for the 1×20 mg/25 mg ramipril-chlorthalidone tablets (Treatment G), the 1×25 mg commercial chlorthalidone tablet (Treatment H), and the 1×25 mg chlorthalidone tablet (Treatment I) are presented in the following table.

TABLE 6
Relative Bioavailability Results for Whole Blood Chlorthalidone for
Group III (Treatments G, H, and I)

| | Treatment G vs. | Treatment G vs. | Treatment I vs. |
|---|---|---|---|
| Parameter | Treatment H | Treatment I | Treatment H |
| Cₘₐₓ | 101.4% (95.39-107.89%) | 98.6% (92.75-104.91%) | 102.8% (96.86-109.19%) |
| (ng/mL) | | | |
| AUC₀₋ₜ | 95.8% (91.46-100.35%) | 96.0% (91.68-100.59%) | 99.8% (95.37-104.36%) |
| (ng · h/mL) | | | |
| AUC_{0-inf} | 92.1% (86.84-97.72%) | 95.4% (89.95-101.23%) | 96.5% (91.14-102.24%) |
| (ng · h/mL) | | | |

Treatment D = 1 × 20 mg/25 mg Ramipril-Chlorthalidone Tablets
Treatment E = 1 × 25 mg Commercial Chlorthalidone Tablet
Treatment F = 1 × 25 mg Chlorthalidone Tablets

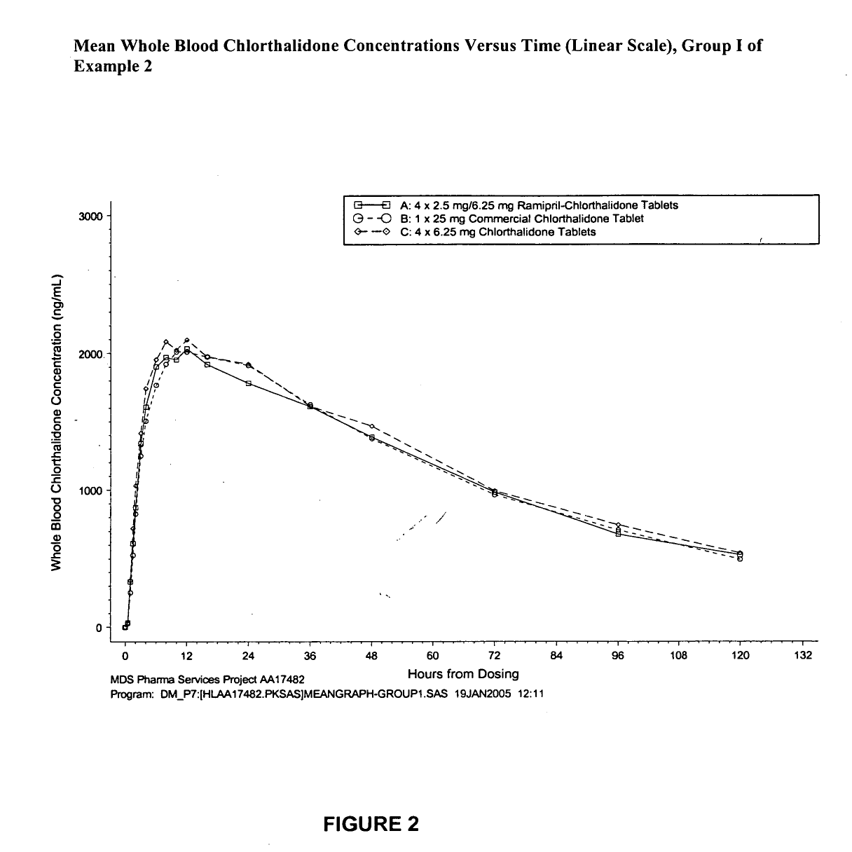

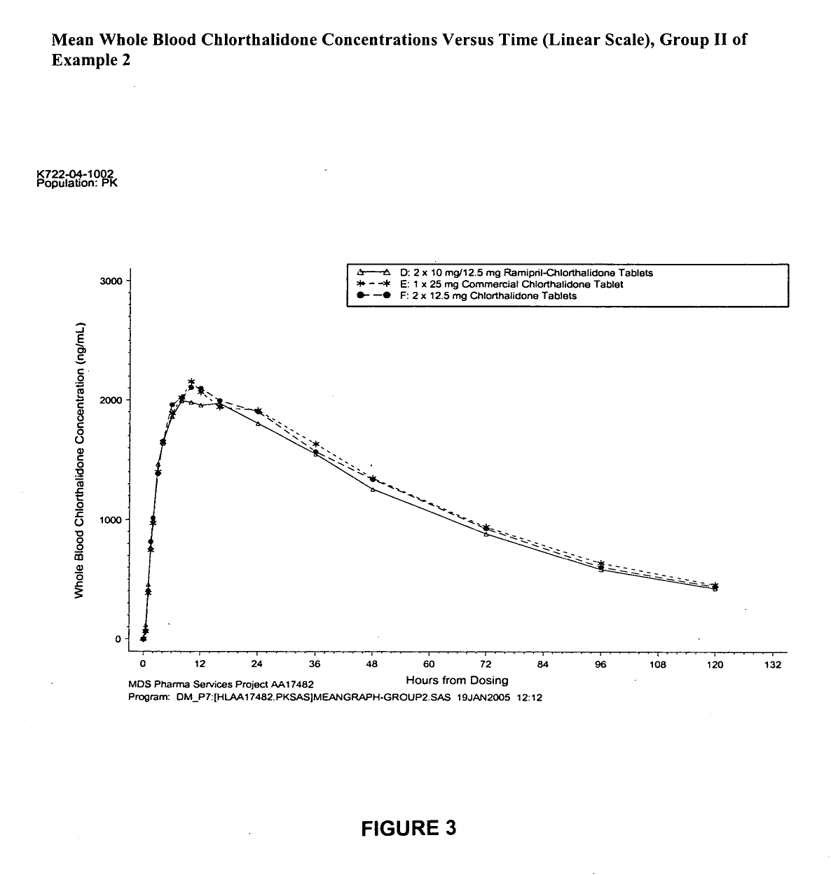

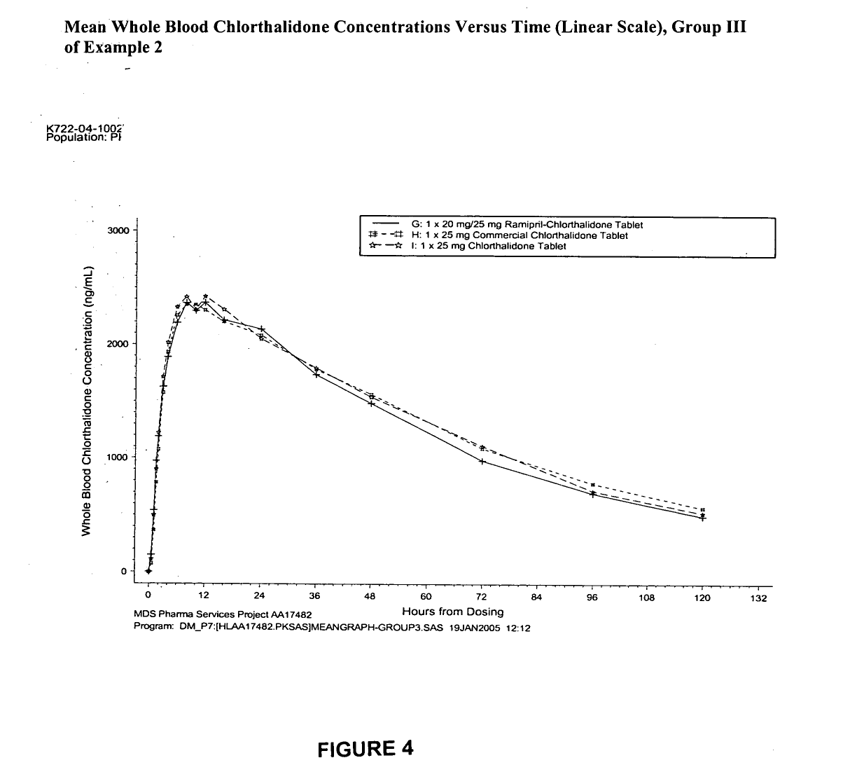

Mean (SD) Plasma Ramipril Concentrations Versus Time for Groups I-III of Example 2 are presented in FIGS. 2-4.

Results

The PK and statistical analyses of the data resulting from this study indicate equivalent chlorthalidone bioavailability between the 2.5 mg/6.25 mg, 10 mg/12.5 mg, and 20 mg/25 mg ramipril-chlorthalidone tablets, and the commercial 25 mg chlorthalidone tablet, as well as the 6.25 mg, 12.5 mg, and 25 mg chlothalidone tablets. The comparison of the chlothalidone tablets and the 25 mg commercial chlorthalidone tablet also indicate equivalent chlorthalidone bioavailability.

### Example 3

### Randomized, Single-Dose, Three-Way Crossover Study to Determine the Bioavailability of Ramipril and Ramiprilat From Ramipril-Chlorthalidone Tablets, Ramipril Tablets, and ALTACE® Capsules in Healthy Volunteers

The study followed a single-dose, open-label, three-period, three-treatment, crossover design and utilized a 2-stage randomization process for treatment group and sequence.

The following treatments were utilized.

Treatment (12 treatments, A-L)

| | | | Ramipril | |
|---|---|---|---|---|
| Treat- | | Ramipril- | Commercial | |
| ment | | Chlorthalidone | Capsule | |
| Group | N | Tablet | (ALTACE ®) | Ramipril Tablet |
| I | 15 | 1 × 2.5 mg/6.25 mg (A) | 1 × 2.5 mg (B) | 1 × 2.5 mg (C) |
| II | 15 | 1 × 5 mg/12.5 mg (D) | 1 × 5 mg (E) | 1 × 5 mg (F) |
| III | 15 | 1 × 10 mg/12.5 mg (G) | 1 × 10 mg (H) | 1 × 10 mg (I) |
| IV | 15 | 1 × 20 mg/25 mg (J) | 2 × 10 mg (K) | 1 × 20 mg (L) |

Subjects were enrolled in Treatment Group I, II, III, or IV and received a total of 3 treatments (ramipril-chlorthalidone tablet, ramipril commercial capsule, and ramipril tablet), which were randomized with respect to sequence. All study drugs were administered as a single dose with 240 mL (8 fluid ounces) of water following an overnight fast. Each treatment was separated by a 3-week washout period.

Sixty (60) subjects were enrolled and 56 subjects completed the study. 56 subjects were included in the pharmacokinetic (PK) analyses.

There were 3 test products examined in this study:

Ramipril/Chlorthalidone combination tablets manufactured by King Pharmaceuticals, Inc.: 2.5 mg/6.25 mg (lot no. 040050, Treatment A), 5 mg/12.5 mg (lot no. 040024, Treatment D), 10 mg/12.5 mg (lot no. 040025, Treatment G), and 20 mg/25 mg (lot no. 040027, Treatment J).

Ramipril commercial capsules (ALTACE®) manufactured by Aventis Pharmaceuticals, Inc. (2.5 mg) or King Pharmaceuticals, Inc. (5 mg and 10 mg): 2.5 mg (lot no. 1063626, Treatment B), 5 mg (lot no. 14888, Treatment E), and 10 mg (lot no. 13053, 1 capsule for Treatment H, and 2 for Treatment K).

Ramipril tablets manufactured by King Pharmaceuticals, Inc.: 2.5 mg (lot no. 040049, Treatment C), 5 mg (lot no. 040019, Treatment F), 10 mg (lot no. 040020, Treatment I), and 20 mg (lot no. 040021, Treatment L).

A single dose of these study drugs was administered with 240 mL of water following an overnight fast. The duration of the treatment was nine (9) weeks including screening.

Pharmacokinetic analyses were performed using plasma concentrations of ramipril and ramiprilat. Pharmacokinetic parameters included the maximum observed plasma concentration (Cₘₐₓ), time of the maximum observed plasma concentration (Tₘₐₓ), and area under the plasma concentration-time curves from time zero to time “t” hours postdose (AUC₀₋ₜ), where t is the last time point with a measurable drug concentration, AUC₀₋₁₂, AUC₀₋₂₄, and AUC₀₋₄₈, where t=12 and 24 for ramipril and t=24 and 48 for ramiprilat.

To evaluate the relative bioavailability, analyses of variance (ANOVA) were performed on the ln-transformed AUC₀₋ₜ, AUC₀₋₁₂, AUC₀₋₂₄, and Cₘₐₓ for ramipril and the ln-transformed AUC₀₋ₜ, AUC₀₋₂₄, and AUC₀₋₄₈, and Cₘₐₓ for ramiprilat. The ANOVA model included terms for treatment, period, sequence, and subject within sequence. A separate ANOVA model was applied to the data from each of the treatment groups. The relative bioavailability of ramipril and ramiprilat from the ramipril-chlorthalidone tablets were compared to that of the ramipril commercial capsules, and to that of the ramipril tablets. In addition, the relative bioavailability of ramipril and ramiprilat from the ramipril tablets were compared to that of the ramipril commercial capsules. For each comparison, the ratios of least-squares means (LSM) and the 90% confidence intervals (CI) were expressed as a percentage relative to the ramipril commercial capsules, or ramipril tablets. A conclusion of equivalent bioavailability was based on the mean ratios and whether the 90% CI for the ln-transformed Cₘₐₓ, AUC₀₋ₜ, AUC₀₋₁₂, AUC₀₋₂₄, and AUC₀₋₄₈ (for ramipril and ramiprilat as appropriate) were within the 80-125% range where the rate and extent of exposure to ramipril and ramiprilat are considered equivalent between the treatments.

A linear relationship between the ln-transformed PK parameters (Cₘₐₓ and AUC) and the ln-transformed dose was fitted using the model ln(Y)=β₀+β lnDose+ε. The 95% CI for the slope between the ln-transformed PK parameters and the ln-transformed dose was calculated for each parameter using the above model. If the 95% CI included 1, then dose proportionality was concluded for the given parameter.

The ratios of the LSM with the 90% CI derived from the analysis of the ln-transformed Cₘₐₓ, AUC₀₋ₜ, AUC₀₋₁₂, and AUC₀₋₂₄ for ramipril and ln-transformed Cₘₐₓ, AUC₀₋ₜ, AUC₀₋₂₄, and AUC₀₋₄₈ for ramiprilat are presented in the following tables.

TABLE 7
Ramipril Relative Bioavailability Results in Plasma for Equivalent
Doses of 2.5, 5, 10, and 20 mg from Ramipril-Chlorthalidone Combination Tablets
Compared to Ramipril Commercial Capsules (ALTACE ®) Ratios of LSM (90% CI)

| | 1 × 2.5 mg/6.25 mg | 1 × 5 mg/12.5 mg | 1 × 10 mg/12.5 mg | 1 × 20 mg/25 mg |
|---|---|---|---|---|
| | tablet (A) | tablet (D) | tablet (G) | tablet (J) vs. |
| | vs. 1 × 2.5 mg | vs. 1 × 5 mg | vs. 1 × 10 mg | 2 × 10 mg |
| Parameter | capsule (B) | capsule (E) | capsule (H) | capsules (K) |
| AUC₀₋ₜ | 110.1% (99.67-121.62%) | 106.9% (98.00-116.61%) | 100.2% (90.71-110.74%) | 92.8% (84.45-101.96%) |
| (ng · hr/mL) | | | | |
| AUC₀₋₁₂ | 105.6% (93.08-119.80%) | 106.9% (95.27-120.04%) | 100.5% (90.42-111.73%) | 88.8% (80.84-98.00%) |
| (ng · hr/mL) | | | | |
| AUC₀₋₂₄ | 106.4% (93.62-120.88%) | 106.8% (94.79-120.28%) | 101.1% (91.11-112.10%) | 87.2% (77.80-97.65%) |
| (ng · hr/mL) | | | | |
| Cₘₐₓ (ng/mL) | 110.4% (94.55-128.80%) | 106.8% (88.84-128.36%) | 94.9% (80.70-111.72%) | 79.2% (69.29-90.55%) |

TABLE 8
Ramiprilat Relative Bioavailability Results in Plasma for Equivalent
Doses of 2.5, 5, 10, and 20 mg from Ramipril-Chlorthalidone Combination Tablets
Compared to Ramipril Commercial Capsules (ALTACE ®) Ratios of LSM (90% CI)

| | 1 × 2.5 mg/6.25 mg | 1 × 5 mg/12.5 mg | 1 × 10 mg/12.5 mg | 1 × 20 mg/25 mg |
|---|---|---|---|---|
| | tablet (A) | tablet (D) | tablet (G) | tablet (J) vs. |
| | vs. 1 × 2.5 mg | vs. 1 × 5 mg | vs. 1 × 10 mg | 2 × 10 mg |
| Parameter | capsule (B) | capsule (E) | capsule (H) | capsules (K) |
| AUC₀₋ₜ | 102.3% (97.97-106.92%) | 101.6% (97.81-105.46%) | 95.3% (89.36-101.70%) | 94.9% (90.83-99.10%) |
| (ng · hr/mL) | | | | |
| AUC₀₋₂₄ | 103.3% (98.12-108.84%) | 101.2% (96.24-106.40%) | 94.2% (87.41-101.52%) | 93.9% (89.30-98.78%) |
| (ng · hr/mL) | | | | |
| AUC₀₋₄₈ | 102.3% (97.97-106.92%) | 101.6% (97.81-105.46%) | 95.3% (89.36-101.70%) | 94.9% (90.83-99.10%) |
| (ng · hr/mL) | | | | |
| Cₘₐₓ (ng/mL) | 102.6% (93.77-112.23%) | 95.9% (87.54-105.02%) | 90.7% (80.05-102.66%) | 88.0% (78.66-98.46%) |

TABLE 9
Ramipril Relative Bioavailability Results in Plasma for Equivalent
Doses of 2.5, 5, 10, and 20 mg from Ramipril-Chlorthalidone Tablets Compared to Ramipril
Tablets Ratios of LSM (90% CI)

| | 1 × 2.5 mg/6.25 mg | 1 × 5 mg/12.5 mg | 1 × 10 mg/12.5 mg | 1 × 20 mg/25 mg |
|---|---|---|---|---|
| | tablet (A) | tablet (D) | tablet (G) | tablet (J) vs. |
| | vs. 1 × 2.5 mg | vs. 1 × 5 mg | vs. 1 × 10 mg | 2 × 10 mg |
| Parameter | capsule (C) | capsule (F) | capsule (I) | capsules (L) |
| AUC₀₋ₜ | 97.9% (88.73-108.10%) | 102.7% (94.18-112.06%) | 95.4% (86.39-105.31%) | 95.6% (87.05-105.09%) |
| (ng · hr/mL) | | | | |
| AUC₀₋₁₂ | 93.9% (82.43-107.04%) | 101.5% (90.45-113.97%) | 102.0% (91.37-113.93%) | 94.9% (86.00-104.61%) |
| (ng · hr/mL) | | | | |
| AUC₀₋₂₄ | 94.4% (82.67-107.71%) | 101.2% (89.88-114.05%) | 102.4% (91.95-114.13%) | 95.9% (86.12-106.82%) |
| (ng · hr/mL) | | | | |
| Cₘₐₓ (ng/mL) | 93.0% (79.74-108.36%) | 101.2% (84.17-121.61%) | 91.9% (78.17-107.95%) | 94.0% (82.23-107.47%) |

TABLE 10
Ramiprilat Relative Bioavailability Results in Plasma for
Equivalent Doses of 2.5, 5, 10, and 20 mg from Ramipril-Chlorthalidone Tablets Compared
to Ramipril Tablets Ratios of LSM (90% CI)

| | 1 × 2.5 mg/6.25 mg | 1 × 5 mg/12.5 mg | 1 × 10 mg/12.5 mg | 1 × 20 mg/25 mg |
|---|---|---|---|---|
| | tablet (A) | tablet (D) | tablet (G) | tablet (J) vs. |
| | vs. 1 × 2.5 mg | vs. 1 × 5 mg | vs. 1 × 10 mg | 2 × 10 mg |
| Parameter | capsule (C) | capsule (F) | capsule (I) | capsules (L) |
| AUC₀₋ₜ | 106.0% (101.51-110.71%) | 101.8% (98.02-105.69%) | 102.8% (96.42-109.62%) | 105.6% (101.12-110.33%) |
| (ng · hr/mL) | | | | |
| AUC₀₋₂₄ | 107.6% (102.22-113.29%) | 101.5% (96.53-106.71%) | 102.8% (95.41-110.68%) | 105.9% (100.66-111.34%) |
| (ng · hr/mL) | | | | |
| AUC₀₋₄₈ | 106.0% (101.51-110.71%) | 101.8% (98.02-105.69%) | 102.8% (96.42-109.62%) | 105.6% (101.12-110.33%) |
| (ng · hr/mL) | | | | |
| Cₘₐₓ (ng/mL) | 108.9% (99.59-119.03%) | 96.2% (87.83-105.37%) | 101.0% (89.31-114.31%) | 109.5% (97.85-122.49%) |

TABLE 11
Ramipril Relative Bioavailability Results in Plasma for Equivalent
Doses of 2.5, 5, 10, and 20 from Ramipril Tablets Compared to Ramipril Commercial
Capsules (ALTACE ®) Ratios of LSM (90% CI)

| | 1 × 2.5 mg | | 1 × 10 mg | |
|---|---|---|---|---|
| | tablet (C) vs. 1 × 2.5 mg | 1 × 5 mg tablet | tablet (I) vs. 1 × 10 mg | 1 × 20 mg |
| | capsule | (F) vs. 1 × 5 mg | capsule | tablet (L) vs. 2 × 10 mg |
| Parameter | (B) | capsule (E) | (H) | capsules (K) |
| AUC₀₋ₜ | 112.4% (101.77-124.18%) | 104.1% (95.40-113.51%) | 105.1% (95.10-116.10) | 97.0% (88.38-106.51%) |
| (ng · hr/mL) | | | | |
| AUC₀₋₁₂ | 112.4% (99.57-126.91%) | 105.3% (93.47-118.68%) | 98.5% (88.33-109.87%) | 93.6% (84.66-103.56%) |
| (ng · hr/mL) | | | | |
| AUC₀₋₂₄ | 112.7% (99.70-127.48%) | 105.5% (93.26-119.26%) | 98.7% (88.65-109.78%) | 90.9% (80.45-102.65%) |
| (ng · hr/mL) | | | | |
| Cₘₐₓ (ng/mL) | 118.7% (101.72-138.56%) | 105.6% (87.82-126.88%) | 103.4% (87.85-121.61%) | 84.3% (73.79-96.21%) |

TABLE 12
Ramiprilat Relative Bioavailability Results in Plasma for
Equivalent Doses of 2.5, 5, 10, and 20 from Ramipril Tablets Compared to Ramipril
Commercial Capsules (ALTACE ®) Ratios of LSM (90% CI)

| | 1 × 2.5 mg | | 1 × 10 mg | |
|---|---|---|---|---|
| | tablet (C) vs.1 × 2.5 mg | 1 × 5 mg tablet | tablet (I) vs. 1 × 10 mg | 1 × 20 mg |
| | capsule | (F) vs. 1 × 5 mg | capsule | tablet (L) vs. 2 × 10 mg |
| Parameter | (B) | capsule (E) | (H) | capsules (K) |
| AUC₀₋ₜ | 96.5% (92.42-100.86%) | 99.8% (96.10-103.61%) | 92.7% (86.92-98.92%) | 89.8% (86.03-93.79%) |
| (ng · hr/mL) | | | | |
| AUC₀₋₂₄ | 96.0% (91.19-101.14%) | 99.7% (94.83-104.84%) | 91.7% (85.06-98.79%) | 88.7% (84.39-93.26%) |
| (ng · hr/mL) | | | | |
| AUC₀₋₄₈ | 96.5% (92.42-100.86%) | 99.8% (96.10-103.61%) | 92.7% (86.92-98.92%) | 89.8% (86.03-93.79%) |
| (ng · hr/mL) | | | | |
| Cₘₐₓ (ng/mL) | 94.2% (86.13-103.08%) | 99.7% (90.99-109.16%) | 89.7% (79.23-101.61%) | 80.4% (71.92-89.84%) |

The PK and statistical analyses of the data resulting from this study indicate comparable bioavailability for both ramipril and ramiprilat (in terms of the Cₘₐₓ and AUC₀₋ₜ LSM) between the 10 mg/12.5 mg ramipril-chlorthalidine tablets and the 10 mg ramipril commercial capsules, between the 5 mg/12.5 mg ramipril-chlorthalidone tablets and the 5 mg ramipril tablets, and between the 20 mg/25 mg ramipril-chlorthalidone tablets and the 20 mg ramipril tablets.

Ramipril and ramiprilat exposure (AUC₀₋ₜ) was comparable between the ramipril-chlorthalidine tablets and both the ramipril commercial capsules and the ramipril tablets at all dose levels, as well as between the ramipril tablets and the ramipril commercial capsules.

In general, the ramipril maximum peak concentrations (Cₘₐₓ) for both the ramipril-chlorthalidone tablets and ramipril tablets tended to be higher than the ramipril commercial capsules following the low dose levels (2.5 mg and 5 mg), but lower following the highest dose level (20 mg). Ramiprilat Cₘₐₓ following ramipril-chlorthalidone tablets and ramipril tablets compared to the ramipril commercial capsules was comparable at the lower dose levels (2.5 mg and 5 mg, and 10 mg), but lower following the high dose level (20 mg).

The ramipril tablets were dose proportional with respect to ramipril AUC₀₋₁₂ and AUC₀₋₂₄. For all formulations, ramipril AUC₀₋ₜ and Cₘₐₓ showed a greater than proportional increase at the 20 mg dose level. All formulations were dose proportional with respect to ramiprilat AUC₀₋₂₄. However, ramiprilat AUC₀₋ₜ and AUC₀₋₄₈ showed a lower than proportional increase with dose level while Cₘₐₓ showed a greater than proportional increase.

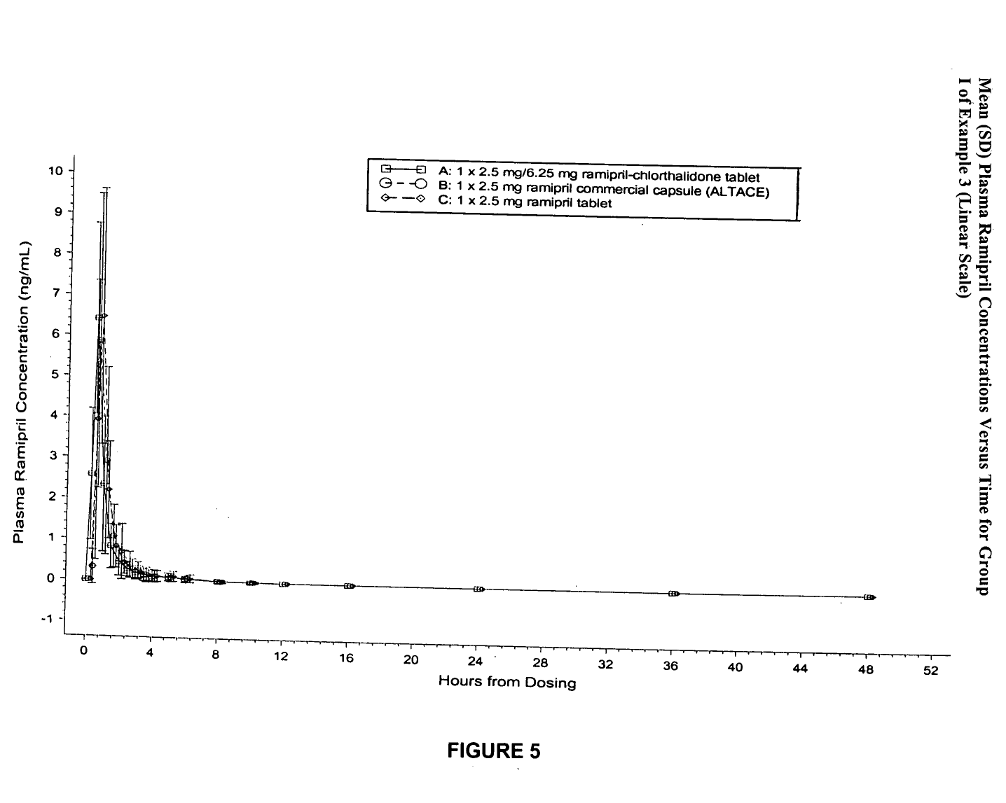

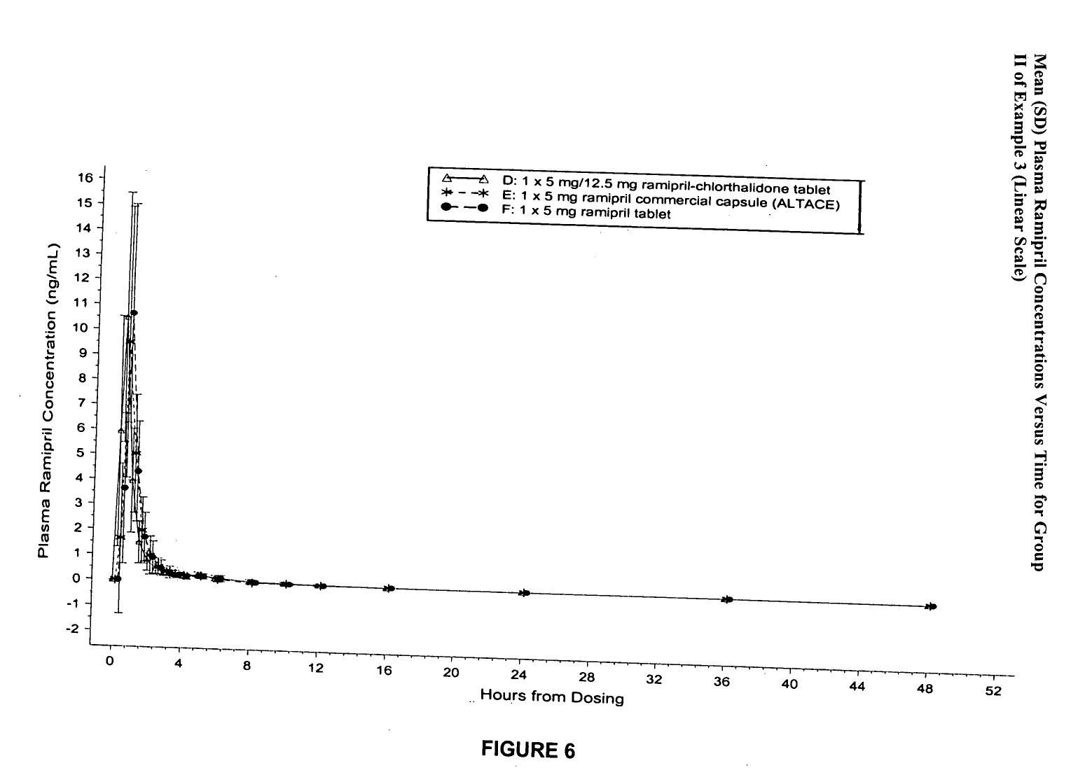

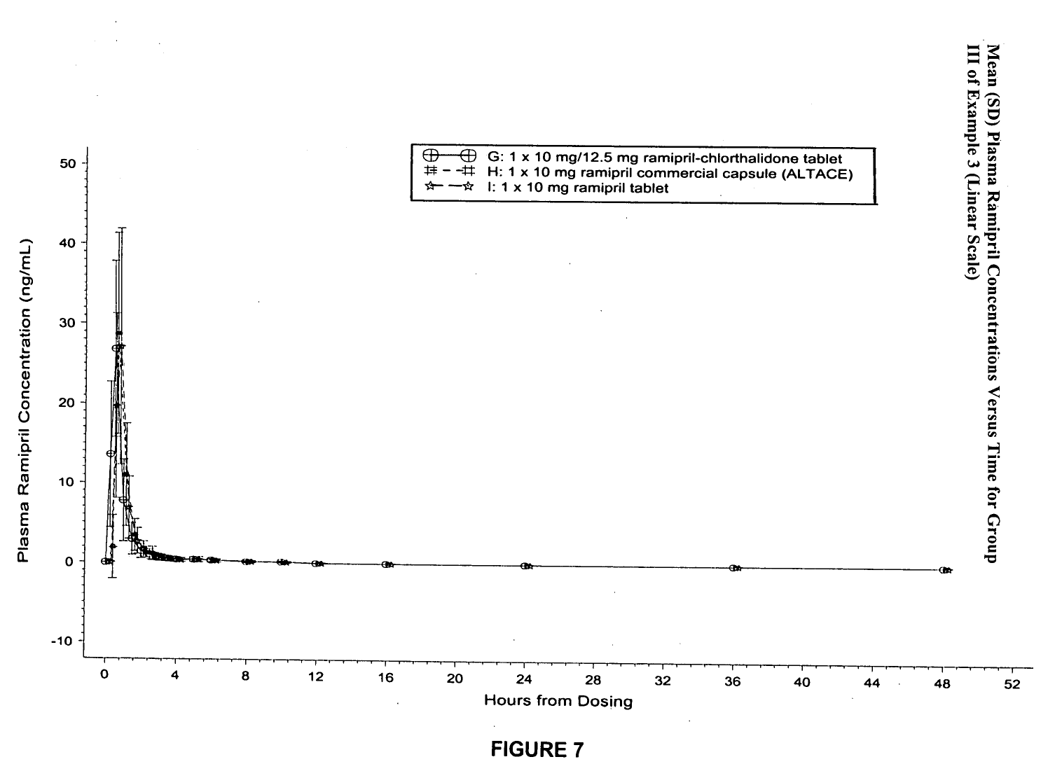

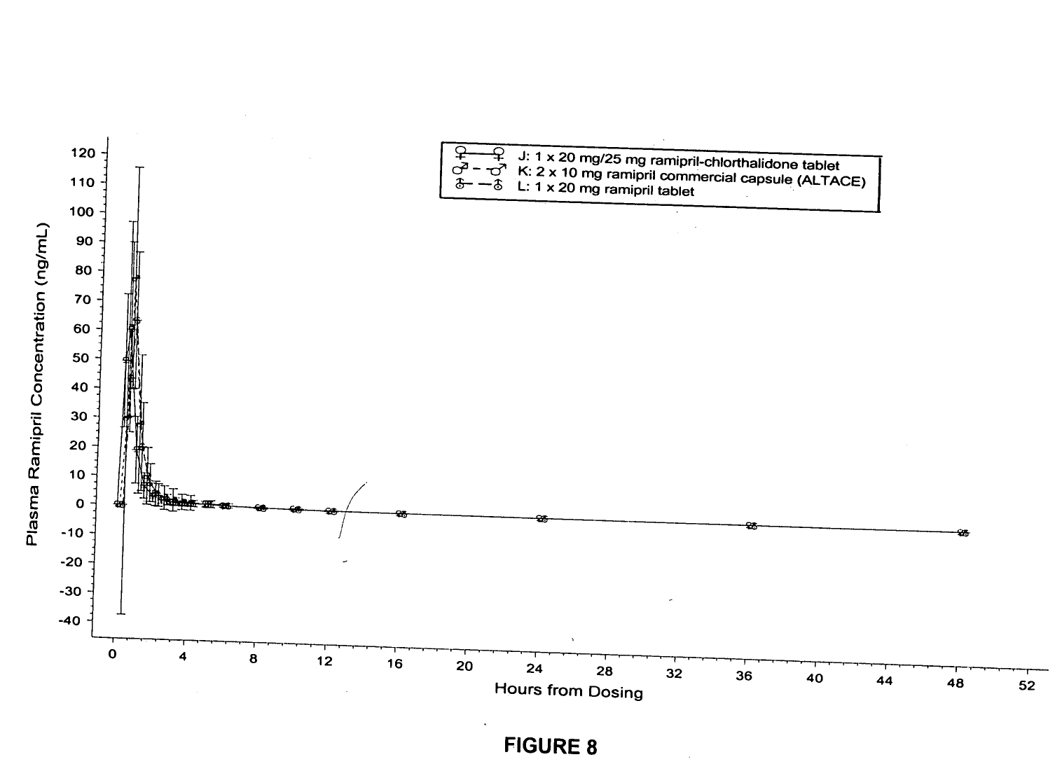

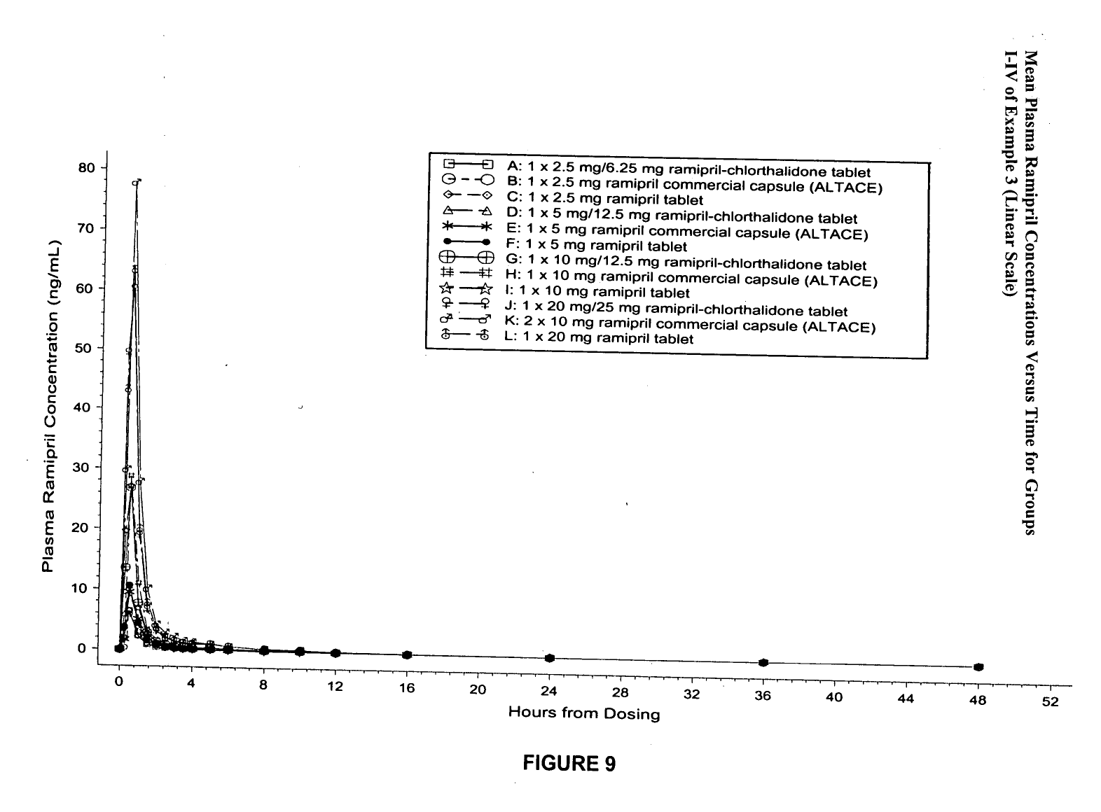

Mean (SD) Plasma Ramipril Concentrations Versus Time for Groups I-IV of Example 3 are presented in FIGS. 5-9.

Results

The pharmacokinetic and statistical analyses of the data resulting from this study also indicate equivalent ramipril bioavailability between the 2.5 mg/6.25 mg, 10 mg/12.5 mg, and 20 mg/25 mg ramipril-chlorthalidone tablets, and the commercial 2.5 mg, 5 mg, and 10 mg ramipril tablets, as well as the 2.5 mg, 5 mg, and 10 mg ramipril tablets.

### Example 4

### Combination of Ramipril and Hydrochlorothiazide

Two batches of ramipril/hydrochlorthiazide combination tablets were made and evaluated for their stability. Batch A was 2.5 mg of GECoated ramipril (ramipril coated with hydroxypropyl methylcellulose) combined with 6.25 mg of hydrochlorothiazde (HCTZ). Batch B was 5 mg of GECoated ramipril (ramipril coated with hydroxypropyl methylcellulose) combined with 25 mg of hydrochlorothiazde.

Samples of batches A and B were exposed to temperature/relative humidity conditions of either 25° C./60% R.H. or 40° C./75% R.H. for up to 6 months. Chemical, physical and dissolution data were collected at one, two, three and six month time periods.

The data from these batches was compared to the data from tablets in batch C which are ramipril only tablets.

TABLE 13
Batch A Stability Study - Chemical Data

| | % Water | | | % Label Claim | % Label Claim |
|---|---|---|---|---|---|
| Time Point | Content | % DKP | % Ramiprilat | HCTZ | Ramipril |
| Initial | 3.7 | 0.19 | 0.07 | 98.8 | 101.2 |
| 1-month | 4.9 | 0.24 | ND | 101.3 | 101.8 |
| 25° C./60% R.H. | | | | | |
| 1-month | 5.3 | 1.19 | ND | 101.9 | 101.8 |
| 40° C./75% R.H | | | | | |
| 2-month | 4.3 | 0.34 | ND | 97.2 | 94.8 |
| 25° C./60% R.H. | | | | | |
| 2-month | 4.4 | 2.31 | ND | 101.3 | 97.4 |
| 40° C./75% R.H | | | | | |
| 3-month | 5.0 | 0.47 | 0.07 | 96.1 | 97.1 |
| 25° C./60% R.H. | | | | | |
| 3-month | 5.0 | 3.31 | 0.16 | 97.7 | 97.1 |
| 40° C./75% R.H | | | | | |
| 6-month | 6.0 | 0.73 | 0.01 | 100.0 | 101.7 |
| 25° C./60% R.H. | | | | | |
| 6-month | 6.4 | 5.24 | 0.03 | 100.0 | 96.8 |
| 40° C./75% R.H | | | | | |

ND = not detected

TABLE 14
Batch A Stability Study - Physical Data

| Time Point | Ave. Wt. (mg) | Thickness (in) | Hardness (kp) | Friability (%) | DT |
|---|---|---|---|---|---|
| Initial | 101.9 | 0.1379 | 11.1 | 0 | 10 sec-13 sec |
| | % RSD = 5.7 | % RSD = 0.7 | % RSD = 20.9 | | |
| 1-month | 100.9 | 0.1373 | 11.4 | 0 | 18 sec-40 sec |
| 25° C./60% R.H. | % RSD = 7.2 | % RSD = 0.6 | % RSD = 22.0 | | |
| 1-month | 101.8 | 0.1383 | 11.0 | 0 | 7 sec-7 sec |
| 40° C./75% R.H | % RSD = 2.1 | % RSD = 0.4 | % RSD = 10.8 | | |
| 2-month | 100.1 | 0.1373 | 10.5 | 0 | NP |
| 25° C./60% R.H. | % RSD = 9.1 | % RSD = 1.0 | % RSD = 23.0 | | |
| 2-month | 100.1 | 0.1379 | 9.4 | 1.3 | NP |
| 40° C./75% R.H | % RSD = 9.1 | % RSD = 1.2 | % RSD = 31.6 | | |
| 3-month | 101.9 | 0.1378 | 11.6 | 0 | NP |
| 25° C./60% R.H. | % RSD = 3.0 | % RSD = 0.4 | % RSD = 14.0 | | |
| 3-month | 101.7 | 0.1393 | 10.4 | 0 | NP |
| 40° C./75% R.H | % RSD = 7.5 | % RSD = 0.7 | % RSD = 21.6 | | |
| 6-month | 99.1 | 0.1371 | 11.0 | 0 | NP |
| 25° C./60% R.H. | % RSD = 10.2 | % RSD = 1.1 | % RSD = 14.3 | | |
| 6-month | 102.7 | 0.1390 | 9.6 | 0 | NP |
| 40° C./75% R.H | % RSD = 2.9 | % RSD = 0.4 | % RSD = 13.3 | | |

RSD = relative standard deviation
DT = disintegration time
NP = not performed

TABLE 15
Batch A Stability Study Dissolution Data

| | | % Label Claim |
|---|---|---|
| | % Label Claim | Ramipril |
| Time Point | HCTZ (mean) | (mean) |
| Initial | 90 | 93 |
| 1-month 25° C./60% R.H. | 91 | 91 |
| 1-month 40° C./75% R.H | 94 | 90 |
| 2-month 25° C./60% R.H. | 101 | 100 |
| 2-month 40° C./75% R.H | 87 | 93 |
| 3-month 25° C./60% R.H. | 91 | 97 |
| 3-month 40° C./75% R.H | 86 | 94 |
| 6-month 25° C./60% R.H. | 98 | 94 |
| 6-month 40° C./75% R.H | 97 | 93 |

TABLE 16
Batch B Stability Study - Chemical Data

| | % Water | | | % Label Claim | % Label Claim |
|---|---|---|---|---|---|
| Time Point | Content | % DKP | % Ramiprilat | HCTZ | Ramipril |
| Initial | 3.4 | 0.19 | 0.13 | 100.6 | 102.7 |
| 1-month | 4.7 | ND | ND | 101.3 | 101.1 |
| 25° C./60% R.H. | | | | | |
| 1-month | 4.7 | 1.05 | ND | 101.3 | 101.1 |
| 40° C./75% R.H | | | | | |
| 2-month | 4.1 | 0.36 | ND | 100.4 | 98.8 |
| 25° C./60% R.H. | | | | | |
| 2-month | 4.0 | 2.02 | ND | 98.8 | 96.5 |
| 40° C./75% R.H | | | | | |
| 3-month | 4.5 | 0.46 | 0.19 | 98.7 | 101.9 |
| 25° C./60% R.H. | | | | | |
| 3-month | 4.8 | 3.08 | 0.21 | 99.2 | 99.3 |
| 40° C./75% R.H | | | | | |
| 6-month | 5.7 | 0.67 | 0.01 | 99.6 | 101.4 |
| 25° C./60% R.H. | | | | | |
| 6-month | 5.5 | 4.60 | 0.13 | 99.1 | 96.6 |
| 40° C./75% R.H | | | | | |

TABLE 17
Batch B Stability Study - Physical Data

| Time Point | Ave. Wt. (mg) | Thickness (in) | Hardness (kp) | Friability (%) | DT |
|---|---|---|---|---|---|
| Initial | 203.8 | 0.1785 | 11.4 | 0 | 5 sec-10 sec |
| | % RSD = 1.0 | % RSD = 0.2 | % RSD = 4.9 | | |
| 1-month | 202.6 | 0.1786 | 11.2 | 0 | 12 sec-76 sec |
| 25° C./60% R.H. | % RSD = 1.0 | % RSD = 0.2 | % RSD = 6.0 | | |
| 1-month | 203.0 | 0.1797 | 10.4 | 0 | 21 sec-29 sec |
| 40° C./75% R.H | % RSD = 1.1 | % RSD = 0.2 | % RSD = 6.4 | | |
| 2-month | 203.9 | 0.1785 | 9.9 | 0 | NP |
| 25° C./60% R.H. | % RSD = 1.0 | % RSD = 0.2 | % RSD = 6.0 | | |
| 2-month | 204.5 | 0.1791 | 9.3 | 0 | NP |
| 40° C./75% R.H | % RSD = 0.9 | % RSD = 0.1 | % RSD = 6.4 | | |
| 3-month | 202.3 | 0.1788 | 10.9 | 0 | NP |
| 25° C./60% R.H. | % RSD = 0.9 | % RSD = 0.1 | % RSD = 4.3 | | |
| 3-month | 202.5 | 0.1804 | 9.7 | 0 | NP |
| 40° C./75% R.H | % RSD = 1.8 | % RSD = 0.2 | % RSD = 9.0 | | |
| 6-month | 202.8 | 0.1789 | 10.5 | 0 | NP |
| 25° C./60% R.H. | % RSD = 1.1 | % RSD = 0.2 | % RSD = 6.3 | | |
| 6-month | 204.3 | 0.1805 | 9.5 | 0 | NP |
| 40° C./75% R.H | % RSD = 1.0 | % RSD = 0.2 | % RSD = 6.5 | | |

RSD = relative standard deviation
DT = disintegration time
NP = not performed

TABLE 18
Batch B Stability Study Dissolution Data

| | | % Label Claim |
|---|---|---|
| | % Label Claim | Ramipril |
| Time Point | HCTZ (mean) | (mean) |
| Initial | 90 | 91 |
| 1-month 25° C./60% R.H. | 91 | 92 |
| 1-month 40° C./75% R.H | 85 | 94 |
| 2-month 25° C./60% R.H. | 95 | 98 |
| 2-month 40° C./75% R.H | 101 | 97 |
| 3-month 25° C./60% R.H. | 84 | 99 |
| 3-month 40° C./75% R.H | 83 | 95 |
| 6-month 25° C./60% R.H. | 100 | 98 |
| 6-month 40° C./75% R.H | 99 | 94 |

TABLE 19
Batch C Chemical stability data

| Sample # | Strength | % LC | | | | | |
|---|---|---|---|---|---|---|---|
| | | Initial | 2 wk 40/75 | 4 wk 40/75 | 8 wk 40/75 | 12 wk 40/75 | 24 wk 40/75 |
| 76F74A | 1.25 mg | 104.4 | 102.6 | 102.7 | 100.4 | 98.33 | 98.6 |
| | | % DKP | | | | | |
| | | Initial | 2 wk 40/75 | 4 wk 40/75 | 8 wk 40/75 | 12 wk 40/75 | 24 wk 40/75 |
| 76F74A | 1.25 mg | 0.31 | 0.7 | 1.1 | 1.92 | 2.6 | 4.7 |
| | | Initial | 2 wk RT | 4 wk RT | 8 wk RT | 12 wk RT | 24 wk RT |
| | | 0.31 | | 0.38 | 0.46 | 0.52 | 0.7 |

Results

Ramipril/hydrochlorothiazide tablets are stable with little decomposition of either ramipril or hydrochlorothiazide. Moreover, the decomposition of ramipril in the combination tablets was similar to the decomposition of ramipril in the combination tablets.

What is claimed:

1. A pharmaceutical composition comprising ramipril, another active agent, and a blending agent, wherein the ramipril is coated by the blending agent, and wherein the blending agent is selected from glyceryl behenate, glyceryl stearate, stearyl alcohol, macrogol stearate ether, palmitostearate, ethylene glycol, polyethylene glycol, stearic acid, cetyl alcohol, lauryl alcohol, amylopectin, poloxymer or combinations thereof.

2. The composition of claim 1, wherein the blending agent is glyceryl behenate.

3. The composition of claim 1, wherein the ramipril is coated ramipril.

4. The composition of claim 1, wherein the composition is a tablet, caplet or capsule.

5. The composition of claim 1, wherein the ramipril is in the amount of about 0.1 mg to 50 mg.

6. The composition of claim 1, wherein the active agent is a diuretic, a angiotensin receptor blocker, an ACE inhibitor, a cholesterol lowering drug, a calcium channel blocker, a beta blocker, a glucose lowering agent, or oral hypoglycemics.

7. The composition of claim 6, wherein the active agent is a diuretic.

8. The composition of claim 7, wherein the diuretic is chlorthalidone or hydrochlorothiazide.

9. The composition of claim 7, wherein the diuretic is in the amount of about 0.1 mg to 50 mg.

10. A method of making a pharmaceutical composition comprising:
a) pre-blending milled ramipril with a blending agent; and
b) combining the product of step a) with another active agent,
wherein the blending agent is glyceryl behenate, glyceryl stearate, stearyl alcohol, macrogol stearate ether, palmitostearate, ethylene glycol, polyethylene glycol, stearic acid, cetyl alcohol, lauryl alcohol, amylopectin, poloxymer or combinations thereof.

11. The method of claim 10, wherein the blending agent is glyceryl behenate.

12. The method of claim 10, wherein the ramipril is coated ramipril.

13. The method of claim 10, wherein the composition is a tablet, caplet or capsule.

14. The method of claim 10, wherein the ramipril is in the amount of about 0.1 mg to 50 mg.

15. The method of claim 10, where in the active agent is a diuretic, a angiotensin receptor blocker, an ACE inhibitor, a cholesterol lowering drug, a calcium channel blocker, a beta blocker, a glucose lowering agent, or oral hypoglycemics.

16. The method of claim 10, wherein the active agent is a diuretic.

17. The method of claim 16, wherein the diuretic is chlorthalidone or hydrochlorothiazide.

18. The method of claim 16, wherein the diuretic is in the amount of about 0.1 mg to 50 mg.

19. A product made by the method of claim 10.

20. A method of treating a cardiovascular disorder in a human comprising administering to the human an effective amount of the composition of claim 1.

21. A method of treating a cardiovascular disorder in a human comprising administering to the human an effective amount of the composition of 19.
